SEQUENCE LISTING (17 sequences)

SEQ ID NO: 1 - protein, 127 aa, Homo sapiens [sequence omitted]

SEQ ID NO: 2 - protein, 127 aa, synthetic construct
APARSPSPSTQPWEHVNAIQEALRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 3 - protein, 117 aa, Homo sapiens
MEFGLSWLFLVAILKGVQCEVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAK

SEQ ID NO: 4 - protein, 20 aa, synthetic construct
GGGGSGGGGSGGGGSGGGGS

SEQ ID NO: 5 - protein, 20 aa, synthetic construct
GGGGAGGGGAGGGGAGGGGA

SEQ ID NO: 6 - protein, 18 aa, synthetic construct
EPKSSDKTHTSPPSPAPE

SEQ ID NO: 7 - protein, 270 aa, synthetic construct
EVQLLESGGGLVQPGGSLRLSCAASGETLDYYAIGWFRQAPGKEREGVSCIASSGGSTNYADSVKGRFTISRDNSKNTVYLQMNSLKPEDTAVYYCAAAVLECRTVVRGYDYWGQGTQVTVSSGGGGSGGGGSGGGGSGGGGSAPARSPSPSTQPWEHVNAIQEALRLLNLSRDTAAEMQETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 8 - protein, 268 aa, synthetic construct
EVQLLESGGGLVQPGGSLRLSCAASGETLDYYAIGWFRQAPGKEREGVSCIASSGGSTNYADSVKGRFTISRDNSKNTVYLQMNSLKPEDTAVYYCAAAVLECRTVVRGYDYWGQGTQVTVSSEPKSSDKTHTSPPSPAPEAPARSPSPSTQPWEHVNAIQEALRLLNLSRDTAAEMQETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 9 - protein, 268 aa, synthetic construct
APARSPSPSTQPWEHVNAIQEALRLLNLSRDTAAEMQETVEVISEMFDLQEPTCLQTRLELYKQGLRGSLTKLKGPLTMMASHYKQHCPPTPETSCATQIITFESFKENLKDFLLVIPFDCWEPVQEEPKSSDKTHTSPPSPAPEEVQLLESGGGLVQPGGSLRLSCAASGETLDYYAIGWFRQAPGKEREGVSCIASSGGSTNYADSVKGRFTISRDNSKNTVYLQMNSLKPEDTAVYYCAAAVLECRTVVRGYDYWGQGTQVTVSS

SEQ ID NO: 10 - protein, 270 aa, synthetic construct
APARSPSPSTQPWEHVNAIQEALRLLNLSRDTAAEMQETVEVISEMFDLQEPTCLQTRLELYKQGLRGSLTKLKGPLTMMASHYKQHCPPTPETSCATQIITFESFKENLKDFLLVIPFDCWEPVQEGGGGSGGGGSGGGGSGGGGSEVQLLESGGGLVQPGGSLRLSCAASGETLDYYAIGWFRQAPGKEREGVSCIASSGGSTNYADSVKGRFTISRDNSKNTVYLQMNSLKPEDTAVYYCAAAVLECRTVVRGYDYWGQGTQVTVSS

SEQ ID NO: 11 - protein, 270 aa, synthetic construct
EVQLLESGGGLVQPGGSLRLSCAASGETLDYYAIGWFRQAPGKEREGVSCIASSGGSTNYADSVKGRFTISRDNSKNTVYLQMNSLKPEDTAVYYCAAAVLECRTVVRGYDYWGQGTQVTVSSGGGGAGGGGAGGGGAGGGGAAPARSPSPSTQPWEHVNAIQEALRLLNLSRDTAAEMQETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 12 - protein, 8 aa, synthetic construct
GETLDYYA

SEQ ID NO: 13 - protein, 8 aa, synthetic construct
IASSGGST

SEQ ID NO: 14 - protein, 16 aa, synthetic construct
AAAVLECRTVVRGYDY

SEQ ID NO: 15 - protein, 9 aa, synthetic construct
ETLDYYAIG

SEQ ID NO: 16 - protein, 16 aa, synthetic construct
IASSGGSTNYADSVKG

SEQ ID NO: 17 - protein, 14 aa, synthetic construct
AVLECRTVVRGYDY

(19) United States
(12) Patent Application Publication
(10) Pub. No.: US 2025/0230210 A1
(43) Pub. Date: Jul. 17, 2025

(54) LONG-ACTING GRANULOCYTE MACROPHAGE-COLONY STIMULATING FACTOR

(71) Applicant: Partner Therapeutics, Inc., Lexington, MA (US)

(72) Inventors: Michael FELDHAUS, Lexington, MA (US); Xiaoniu MIAO, Zhuhai City, Guangdong Province (CN); Chao WANG, Zhuhai City, Guangdong Province (CN); Yi LUO, Zhuhai City, Guangdong Province (CN); Yuefeng ZOU, Zhuhai City, Guangdong Province (CN)

(21) Appl. No.: 18/854,328

(22) PCT Filed: Mar. 28, 2023
(86) PCT No.: PCT/US23/65047

§ 371 Date: Oct. 4, 2024

Related U.S. Application Data
(60) Provisional application No. 63/329,039, filed on Apr. 8, 2022.

(51) Int. Cl. C07K 14/535; A61K 38/00; C07K 16/18

(52) U.S. Cl. CPC C07K 14/535; C07K 16/18; A61K 38/00; C07K 2317/24; C07K 2317/52; C07K 2317/55; C07K 2317/569; C07K 2317/92; C07K 2317/94; C07K 2319/00

(57) ABSTRACT

The present disclosure relates generally to compositions and methods related to long-acting and granulocyte-macrophage colony-stimulating factor (GM-CSF) with improved pharmacokinetics.

### CROSS REFERENCE TO RELATED APPLICATION

This application claims priority to U.S. Provisional Patent Application No. 63/329,039, filed Apr. 8, 2022, the entire contents of which is hereby incorporated by reference for all purposes.

### FIELD

The present disclosure relates, inter alia, to compositions and methods related to long-acting and granulocyte-macrophage colony-stimulating factor (GM-CSF) with improved pharmacokinetics.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: PNR-009PC 127114-5009-PC Sequence Listing.xml”; date recorded: Mar. 23, 2023; file size: 20,480 bytes).

### BACKGROUND

Colony Stimulating Factor, CSF, refers to a family of four glycoproteins that control and coordinate cell production by widely scattered deposits of marrow cells. These include: Granulocyte-Macrophage CSF (GM-CSF), Granulocyte colony CSF (G-CSF), Macrophage colony CSF (M-CSF) and multipotential colony-stimulating factor (IL-3). These lymphokines can induce progenitor cells found in the bone marrow to differentiate into specific types of mature blood cells. The particular type of mature blood cell that results from a progenitor cell depends upon the type of CSF present. See Metcalf D. Cancer Immunol Res. 2013, 1(6): 351-356.

GM-CSF is a hematologic growth factor that regulates the production, migration, proliferation, differentiation and function of hematopoietic cells. In response to inflammatory stimuli, GM-CSF is released by various cell types including T lymphocytes, macrophages, fibroblasts and endothelial cells. GM-CSF then activates and enhances the production and survival of neutrophils, eosinophils, and macrophages. Native GM-CSF is usually produced near the site of action where it modulates in vitro proliferation, differentiation, and survival of hematopoietic progenitor cells, but is present in circulating blood in only picomolar concentrations (10⁻¹⁰ to 10⁻¹² M). See Alexander W S. Int Rev Immunol. 1998, 16:651-682; Gasson J C. Blood. 1991, 77:1131-1145; Shannon M F et al. Crit Rev Immunol. 1997, 17:301-323, Barreda D R et al. Dev Comp Immunol. 2004, 28:509-554 and Metcalf D. Immunol Cell Biology. 1987, 65:35-43.

Human GM-CSF (hGM-CSF) is synthesized as a 144 amino acid residue precursor protein with a 17 amino acid signal peptide. This precursor protein is processed to yield a 127 amino acid mature protein with a predicted molecular mass of 14.4 kDa. It has two disulfide linkages that migrates as a broad band of 15-30 kDa due to glycosylation and sialylation. N-linked glycans are located at Asn²⁷ and Asn³⁷ and multiple potential sites for O-linked glycosylation exist at Ser5, Ser7, Ser9 and Ser10 but the extent of glycan structures at these sites have not been unambiguously determined. The glycosylation patterns of GM-CSF have been observed to influence its activity, receptor binding, immunogenicity, and half-life. See Lee F. et al. Proc Natl Acad Sci USA Biochem. 1985. 82: 360-4364; Miyatake S. et al. EMBO J. 1985. 4: 2561-2568. Cebon J et al. J Biol. Chem. 1991. 265, 4483-4491; Zhang Q et al. Proc. Natl. Acad. Sci. 2014, 2885-2890.

Recombinant human granulocyte-macrophage colony-stimulating factor (rhu GM-CSF) has been approved by the FDA for the treatment of neutropenia, blood dyscrasias and malignancies like leukemia in combination with chemotherapies. In the clinic, GM-CSF used for treatment of neutropenia and aplastic anemia following chemotherapy greatly reduces the risk of infection associated with bone marrow transplantation. Its utility in myeloid leukemia treatment and as a vaccine adjuvant is also well established. See Dorr R T. Clin Therapeutics. 1993. 15(1):19-29; Armitage J O. Blood 1998, 92:4491-4508; Kovacic J C et al. J Mol Cell Cardiol. 2007, 42:19-33; Jacobs P P et al. Microbial Cell Factories 2010, 9:93.

Although there are five classes of heterologous protein production platforms, including bacteria, yeasts, plants, insect cells, and mammalian cells, more than 50% of currently marketed biopharmaceuticals are produced in mammalian cell lines. This is in part due to the inability of the remaining four classes to modify glycoproteins with human-like oligosaccharides. This is of importance as protein-bound glycans influence circulation half-life, tissue distribution, biological activity and immunogenicity.

The GM-CSF expression system influences the pharmacokinetics properties, biological activity and clinical toxicity of GM-CSF. In the clinic, GM-CSF has been produced in Chinese hamster ovary cells (CHO-GM, regramostim), Escherichia coli (E. coli-GM, molgramostim), or yeast (Yeast-GM, sargramostim). See Dorr R T. Clin Therapeutics. 1993. 15(1):19-29; Walsh G. Nat Biotechnol. 2006, 24:769-776; Jacobs P P et al. Nat Protoc. 2009, 4:58-70; Jacobs P P et al. Microbial Cell Factories 2010, 9:93; Walsh G. Nat Biotechnol. 2018, 36(12): 1136-1145. During the manufacture of GM-CSF, four major GM-CSF isoforms/species are present: “hyper-glycosylated” (˜50 kDa), N- and N-+O-glycosylated (˜20 kDa), 0-glycosylated (˜16 kDa) and the non-glycosylated (˜15 kDa) species. Glycosylated GM-CSF is obtained via recombinant technologies using yeast (sargramostim) or Chinese hamster ovary (CHO) cell (regramostim) technologies, which yield complex mixtures of glycoforms. Human cell derived-GM-CSF is also like-wise heterogeneously glycosylated, However, GM-CSF remains unglycosylated when it is made in E. coli. The glycan heterogeneity reflects a lack of specificity in CHO-cell posttranslational glycosylation. See Dorr R T. Clinical Therapeutics/vol. 15, NO. 1, 1993; Zhang Q et al. PNAS. 2014. 111(8): 2885-2890.

N-glycosylation occurs when glycans are added to the nitrogen of asparagine (Asn) or Arginine (Arg) side chains, whereas O-glycosylation occurs when glycans are added to the hydroxyl oxygen side chains of serine (Ser) or threonine (Thr) or tyrosine (Tyr) amino acids. Asparagine residues typically require a consensus sequence of Asn-Xxx-Ser/Thr/Cys, where Xxx can be any amino acid except proline, to be N-glycosylated. The rhu GM-CSF protein sequences for sargramostim contain two consensus sequences, Asn-Leu-Ser (NLS) and Asn-Glu-Thr (NET), where the protein can potentially be glycosylated. During manufacture of sargramostim both N-glycosylation sites, Asn²⁷ and Asn³⁷, are glycosylated in some GM-CSF species present in the fermenter. The Thr³⁹ (or Ser³⁹ or Cys³⁹) residue is an important anchor to enable glycosylation at Asn²⁷ or Asn³⁷. Species with N-oligosaccharides attached at asparagine residue 37 are referred to as “hyper-glycosylated” species (30-100 kDa) because these glycoforms contain up to several hundreds of mannose residues. Hypermannosylation only occurs at N37.

The final product of sargramostim (LEUKINE) consists of N-glycosylated and O-glycosylated glycoforms along with non-glycosylated forms. Thus, sargramostim has heterogeneity in its glycoform profile which has been very consistent throughout its licensed history. Nearly half of the GM-CSF protein in sargramostim is non-glycosylated and slightly less than a third is fully N-glycosylated. Most if not all of the N-glycosylated species are also 0-glycosylated. The highly branched N-linked oligosaccharide structure overlays a significant portion of the molecule including the H1 and H5 alpha-helices without obstructing the receptor binding site as well as some of the H6 alpha-helix and C-terminal random coil. Although the carbohydrate component of recombinant GM-CSF differs based on the cellular source, glycosylation is not required for receptor binding or for in vitro and in vivo biological activity. Yeast rhu GM-CSF and non-glycosylated E. coli-derived rhu GM-CSF have comparable specific activities measured by both receptor affinity and cell proliferation specific activity. See Urdal and Park, 1988; Metcalf D. Cancer 1990: 65:2185-2195.

Endogenous proteins in humans are degraded in vivo at different rates which may vary widely depending on their functions. For instance, hemoglobin lasts for the entire life-time of an erythrocyte and histones have a half-life measured in years while other proteins like ornithine decarboxylase (11 min half-life) have very rapid degradation rates. See Thomas E Creighton (1993). “Chapter 10—Degradation”. Proteins: Structures and Molecular Properties (2nd ed.). W H Freeman and Company. pp. 463-473. ISBN 0-7167-2317-4. In eukaryotes, most short-lived proteins in the intracellular space are degraded by the ubiquitin-proteasome pathway (UPP). See Kybuczkova L, et al. J Cell Mol Med. 2014. 18: 947-961. The UPP plays a central role in cellular homeostasis in the processing and degradation of proteins, including those that regulate cell-cycle progression, apoptosis, and DNA repair. See Heinemeyer W, et al. J Biol Chem. 1997. 272: 25200-25209. Lysosomes also remove unwanted proteins in the cytoplasm and from outside the cell. Material from outside the cell is taken-up through endocytosis, while material from the inside of the cell is digested through autophagy See Settembre C, et al. Nature Reviews Molecular Cell Biology. 2013.14 (5): 283-96. GM-CSF has been shown to have a half-life that can extend from 50 to 85 min. See Cebon et al. Blood. 1988. 72: 1340-1347; Stagg et al 2004. The in vivo biological activity of naturally occurring and recombinant GM-CSF is largely dependent upon their bioavailability. See Dorr R T. Clinical Therapeutics/vol. 15, NO. 1, 1993.

Glycosylation has been reported to stabilize a variety of proteins. Some charged terminal glycans (e.g., sialic acid) have been found to be critical in regulating the circulatory half-life of glycoproteins. There are also several reports where glycosylation is essential in maintaining the conformational stability of proteins against pH denaturation, for example in G-CSF (GRANOCYTE). See Nissen C. Eur J Cancer. 1994; 30A (Suppl 3):S12-14; Ono M. Eur J Cancer. 1994; 30A(Suppl 3):S7-11. Increased pH stability can be also artificially engineered into proteins, for example through the glycation of penicillin G acylase. The half-life for the glycated version of this protein was increased 13-fold at pH 3 and 7-fold at pH 10 when compared to the non-glycated protein. See Masarova J, Biotechnol Appl Biochem. 2001; 34(Pt 2):127-133. It has also been shown that both the degree of glycosylation and the glycan size can increase a protein's inactivation half-life but with significantly greater magnitude of kinetic stabilization brought about at increasing glycan size. See Solá R J, Al-Azzam W, Griebenow K. Biotechnol Bioeng. 2006; 94(6):1072-1079; Sola R J, Cell Mol Life Sci. 2007; 64(16):2133-2152; Sola R J et al. J Pharm Sci. 2009. 98(4): 1223-1245.

The bioavailability of therapeutic proteins (biotherapeutic or biologic) in humans can also be influenced by the route of administration of the drug. See Dorr R T, Clinical Therapeutics/vol. 15, NO. 1, 1993. Immediately following intravenous (IV) administration nearly all of the therapeutic protein is available, but then may be degraded over time by proteases present in blood plasma. In some cases, subcutaneous (SC) administration has provided prolonged exposure to proteins with short elimination half-lives by maintaining high plasma concentrations for longer periods and can be better tolerated compared to IV administration. Thus, absorption of a biotherapeutic after SC administration may be influenced by its fate in the extra-cellular matrix (ECM) including the possibility of incomplete bioavailability. See Hale G, et al. Blood. 2004.104(4):948-955. Transport of the different isoforms present in sargramostim into the systemic circulation may favor different routes because of their different molecular sizes.

The GM-CSF expression system can influence the pharmacokinetic parameters, with the extent of glycosylation affecting GM-CSF half-life, distribution, and elimination. A relationship between the extent of GM-CSF glycosylation and its distribution, clearance, and activity has been reported using a rat model. The specific activity of GM-CSF measured in vitro was found to be significantly decreased in the largest, most fully glycosylated forms of the protein relative to the smaller, less heavily glycosylated molecules. The effective half-life of GM-CSF in the bloodstream of a rat following a single intravenous bolus injection was shown to be significantly increased by the addition of N-linked carbohydrate. The clearance of GM-CSF in the rat follows biphasic kinetics, and it is the first or c˜phase that is prolonged by the carbohydrate modification. See Donohue R E, et al. Cold Spring Harbor Labs. 1986. 51: 685-692 and Dorr R T Clinical Therapeutics. 1993. 15(1): 19-29. Another way to influence pharmacokinetic parameters is to conjugate target proteins, like GM-CSF, to albumin. This can harness the long half-life and targeting abilities of albumin to enhance the biostability of the target protein. See Chuang Y-M et al. Cell Mol Immunol. 2020.

Anti-serum albumin domain antibodies have also been shown to extend the half-lives of short-lived drugs. See Holt L J et al. Protein Eng Des Sel. 2008. 21(5):283-8. However, the large size of conventional antibodies can hamper delivery of target proteins or drugs to cells in vivo. Nanobodies (15 kDa) and nanobody-based human heavy chain antibodies (75 kDa) can overcome these limitations. Camelids naturally produce antibodies composed only of heavy chains in which the target recognition module is composed of a single variable domain (VHH or Nb). Advantageous features of nanobodies include their small size, high solubility, high stability, and excellent tissue penetration in vivo. See Bannas P et al. Front Immunol. 2017. The in vivo half-life of molecules, for example anti-tumor nanobodies, can be extended by fusing one or more antitumor nanobodies to an albumin-specific nanobody. See Roovers R C et al. Int J Cancer. 2011. 129(8):2013-24; Zhu Y et al. Sci Rep (2017) 7(1):2602; Bannas P et al. Front Immunol. 2017. It stand to reason that increasing the half-life and circulation time of GM-CSF-based therapies could decrease the dose and simplify the treatment-regime of the drug administered to patients. This could have positive effects on quality of patient care, patient compliance and reduced costs of medical care.

There remains a need for GM-CSF molecules with improved pharmacokinetics with increased half-life and increased circulation time.

### SUMMARY

Accordingly, the present disclosure relates to long-acting forms of GM-CSF, which, inter alia, provide improved pharmacokinetics and pharmacodynamics, relative to wild type GM-CSF or sargramostim.

In aspects, there is provided a fusion or chimeric protein, the protein comprising a recombinant human GM-CSF protein, a linker and a humanized single domain antibody.

In aspects, the recombinant human GM-CSF protein of the present composition comprises an amino acid sequence having at least about 97% identity with SEQ ID NO: 1 or SEQ ID NO: 2 and optionally having a substitution or deletion at position N37 or a position corresponding thereto, e.g. the amino acid at position N37 or a position corresponding thereto may be substituted to a polar and neutral of charge hydrophilic amino acid, such as glutamine (Q), serine (S), threonine (T), proline (P), and cysteine (C).

In aspects, the humanized single domain antibody of the present composition comprises a heavy chain with three CDRs, e.g. as defined as IMGT nomenclature or Kabat nomenclature.

### IMGT Nomenclature:

CDR-H1:
(SEQ ID NO: 12)
GETLDYYA
CDR-H2:
(SEQ ID NO: 13)
IASSGGST
CDR-H3:
(SEQ ID NO: 14)
AAAVLECRTVVRGYDY

### Kabat CDR Definition:

CDR-H1:
(SEQ ID NO: 15)
ETLDYYAIG
CDR-H2:
(SEQ ID NO: 16)
IASSGGSTNYADSVKG
CDR-H3:
(SEQ ID NO: 17)
AVLECRTVVRGYDY.

In aspects, the humanized single domain antibody of the present composition comprises a heavy chain with three CDRs of SEQ ID NO: 12, SEQ ID NO: 13, and SEQ ID NO: 14, or variants having one or more substitutions or deletions (e.g. 1, or 2, or 3, or 4). In aspects, the humanized single domain antibody of the present composition comprises a heavy chain with three CDRs of SEQ ID NO: 15, SEQ ID NO: 16, and SEQ ID NO: 17, or variants having one or more substitutions or deletions (e.g. 1, or 2, or 3, or 4). In aspects, the humanized single domain antibody of the present composition comprises an amino acid sequence having at least about 70%, or at least about 75%, or at least about 80%, or at least about 85%, or at least about 90%, or at least about 95%, or at least about 98%, or at least about 99% identity with SEQ ID NO: 3 and having substitutions or deletions within a non-human llama antibody framework to enable humanization of the non-human antibody. The CDRs are defined as IMGT nomenclature. CDRs can also be defined by Kabat nomenclature.

### IMGT Nomenclature:

CDR-H1:
(SEQ ID NO: 12)
GETLDYYA
CDR-H2:
(SEQ ID NO: 13)
IASSGGST
CDR-H3:
(SEQ ID NO: 14)
AAAVLECRTVVRGYDY

### Kabat CDR Definition:

CDR-H1:
(SEQ ID NO: 15)
ETLDYYAIG
CDR-H2:
(SEQ ID NO: 16)
IASSGGSTNYADSVKG
CDR-H3:
(SEQ ID NO: 17)
AVLECRTVVRGYDY

In aspects, the fusion or chimeric protein, the protein comprising a recombinant human GM-CSF protein is fused to a humanized single domain antibody via a linker.

In embodiments, the present composition comprises a plurality of molecular forms, e.g. non-glycosylated, 0-glycosylated, N-glycosylated and/or N+O glycosylated forms. In embodiments, the present fusion or chimeric protein is substantially free of hyperglycosylated, e.g. hypermannosylated forms.

In embodiments, there is also provided nucleic acid molecules encoding the present fusion or chimeric protein (e.g. a codon-optimized sequence). In embodiments, there is also provided a human host cell expressing the nucleic acid molecule encoding the present fusion or chimeric protein optionally a Chinese hamster ovary (CHO) cell. In embodiments, there is also provided a pharmaceutical composition comprising the present fusion or chimeric protein and a pharmaceutically acceptable excipient or carrier.

In aspects, there is provided a method of treating a patient or subject who is undertaking or has undertaken a cancer therapy, or who is undertaking or has undertaken a bone marrow transplant, or who had been acutely exposed to myelosuppressive doses of radiation (e.g. Acute Radiation Sickness or Acute Radiation Syndrome (ARS)) and/or has a Radiation Combined Injury (RCI); the method comprising administering to the patient a therapeutically effective amount of the pharmaceutical compositions described herein.

In aspects, there is a provided a method of treating a neurodegenerative disease, e.g. without limitation Alzheimer's disease, Parkinson's disease, progressive supranuclear palsy (PSP), multiple system atrophy (MSA), Lewy body dementia, Parkinson's disease dementia epilepsy, stroke, Huntington's Chorea, cerebral hypoxia, multiple sclerosis, amyotrophic lateral sclerosis (ALS), and peripheral neuropathy, and the subject in need thereof having been treated with the fusion or chimeric protein, or pharmaceutical composition thereof, described herein.

In aspects, there is provided a method of treating a fungal infection, e.g. without limitation an invasive fungal infection optionally selected from infection by candidiasis and/or mucormycosis, and the subject in need thereof having been treated with the fusion or chimeric protein, or pharmaceutical composition thereof, described herein.

In aspects, there is provided a method of treating an autoimmune disease, optionally selected from Crohn's disease and/or ulcerative colitis (UC), irritable bowel disease (IBD) and/or other GI inflammatory diseases, a patient or subject in need thereof having been treated with the fusion or chimeric protein, or pharmaceutical composition thereof, described herein.

In embodiments, the present fusion or chimeric protein is functionally similar to sargramostim. In embodiments, the fusion or chimeric protein has enhanced pharmacokinetics as compared to a recombinant human GM-CSF. In embodiments, the fusion or chimeric protein has enhanced half-life as compared to a recombinant human GM-CSF. In embodiments, the fusion or chimeric protein has enhanced pharmacodynamics as compared to a recombinant human GM-CSF.

### BRIEF DESCRIPTION OF DRAWINGS

FIG. 1A illustrates a structural representation of heavy chain IgG (hclgG).

FIG. 1B shows the yeast surface display system used for the screening of antigen-binding scaffold libraries. VHHs were fused via its C-terminal to the N-terminus of Aga2p. Surface expression can be detected by using fluorescently labelled antibodies that bind the Myc tag.

FIG. 1C illustrates a graph that shows the half-life extension of an Fab fragment facilitated by VHH in a murine model. The various lines on the graph illustrate the various constructs. Bi-340-352 is the anti-albumin VHH half-life extension molecule as a fusion partner with an Fab fragment. Bi-340-356 is an irrelevant VHH fusion with the same Fab fragment showing no half-life extension.

FIG. 2 illustrates the four constructs that were cloned and transiently expressed in CHO cells with or without the secretion leader sequences. The secretion leader sequence was the same for all 4 constructs. The 4 constructs include: VHH-G4Sx4 linker-GMCSF, VHH-hinge linker-GMCSF, GMCSF-hinge linker-VHH and GM-CSF-G4Sx4 linker-VHH. The figure also illustrates the VHH-G4Ax4 linker-GM-CSF with or without the secretion leader sequence.

FIGS. 3A-B show graphs for the results of TF-1 assays with and without human serum albumin (HSA) for the various fusion or chimeric proteins, CHO-produced GM-CSF (ATUM CHO N37Q) and LEUKINE/QCRS-GMCSF (shown as individual graph lines). BDS LEUKINE/QCRS GMCSF refers to wild type GM-CSF, SEQ ID NO: 2, without either of T39A or N37Q amino acid substitutions within the sequence. CHO N37Q is the CHO produced GMCSF variant used in the VHH constructs. H-G-GS-H is the GMCSF-G4Sx4-VHH and the H-H-GS-G is the VHH G4Sx4-GMCSF molecule. The TF1 cell based assay was repeated with the addition of the HSA at 40 ug/ml (physiologic concentration in blood) or without the addition of HSA to cell assay.

FIG. 4A illustrates an example spectrograph used to verify N-terminus cleavage heterogeneity as well as glycosylation of the VHH-G4Sx4-GMCSF variant (VHH-G4Sx4-GMCSF*). High resolution liquid chromatography-mass spectrometry (LC-MS) (Vanquish UPLC Q Exactive, Thermo) was used to determine the intact molecular weight and glycan profile. The figure show glycan analysis by mass spectrometry and specifically glycan occupancy of 6 stable CHO clones making the VHH-G4A GMCSF*. 100 μg sample was diluted by deionized water to 2 mg/mL, then injected to LC-MS. Data was analyzed and deconvoluted by BioPharmaFinder software to obtain the intact protein molecular mass. The mass spectrometry analysis also shows the known glycosylation sites in GMCSF: Ser7 Ser10 and N27 were also glycosylated in all the VHH-G4Sx4-GMCSF variant and similar to the CHO produced GMCSF* used in the fusion proteins.

FIG. 4B shows a graph depicting the results of a TF-1 assay. VHH-G4A GMCSF* produced from the six stable CHO clones were assayed in a TF-1 cell based assay.

FIGS. 5A-C show graphs to illustrate Surface Plasmon Resonance (SPR) assessment of VHH binding to three species of albumin, including human, murine and rhesus monkey. To assess binding, the VHH were cloned onto a human IgG1 Fc and hinge for ease of purification and analysis. Binding affinities of the VHH-Fc constructs were determined by BLI using an Octet 384 (Pall Life Sciences). The VHH-Fc constructs were loaded onto AHC sensors prior to exposure to the monomeric targets, human, mouse or rhesus albumin in solution to determine association and dissociation. In the case of the pH assessment, the binding reaction and dissociation buffer were at the described pH. Data acquisition was performed at 30° C.

FIGS. 6A-D show graphs to illustrate Surface Plasmon Resonance (SPR) assessment of VHH binding to human albumin at various pH at 30° C. The graphs show the response units (target binding to the VHH-Fc on the chip sensor) on the Y axis and time on the X axis Data (time and response units) was analyzed using the ForteBio Data Analysis Software.

FIGS. 7A-C shows graphs illustrating the half-life (t_{1/2}) or pharmacokinetics of the fusion or chimeric protein, VHH-GA-GMCSF* compared to CHO-produced GM-CSF and LEUKINE following intravenous injection with 10 ug/Kg of the listed form of GMCSF. A single rhesus monkey was intravenously injected with 10 ug/Kg of the listed form of GMCSF. Blood draws were taken at 12 min, 30 min, 1, 2, 4, 8, 24, 48 and 72 hours post injection, and analyzed for cell content and the concentration of the construct in the blood determined by a construct specific ELISA. VHH-GA-GMCSF* shows a t_{1/2} approximately 17-fold greater than Leukine or the CHO produced form of GMCSF used in the VHH-GA-GMCSF construct. CHO control is GMCSF variant used on the VHH construct (GMCSF R23L and N37Q) made in CHO cells to maintain similar glycosylation patterns. The yeast control is LEUKINE.

FIGS. 8A-E show graphs for the number of neutrophils, monocytes, eosinophils, total WBC and platelets determined following intravenous (IV) injection of fusion or chimeric protein construct as compared to CHO and LEUKINE (yeast-grown GM-CSF). CHO control is GMCSF variant used on the VHH construct (GMCSF R23L and N37Q) made in CHO cells to maintain similar glycosylation patterns.

### DETAILED DESCRIPTION

The present disclosure is based, in part, on a fusion or chimeric protein comprising a recombinant human GM-CSF protein, a linker and a humanized single domain antibody, wherein the composition yields a functional GM-CSF has an enhanced half-life, pharmacokinetics and pharmacodynamics.

### Compositions of GM-CSF

In an aspect, there is provided engineered fusion or chimeric proteins.

In embodiments, the engineered GM-CSF used in the practice of the disclosure includes any pharmaceutically safe and effective GM-CSF, or any derivative thereof having the biological activity of GM-CSF, optionally with the present substitutions and/or deletions.

In an embodiment, the engineered GM-CSF used in the practice of the subject methods is derived from recombinant human GM-CSF (rhu GM-CSF), such as sargramostim (LEUKINE). Sargramostim is a biosynthetic, yeast-derived, recombinant human GM-CSF, having of a single 127 amino acid glycoprotein that differs from endogenous human GM-CSF by having a leucine instead of an Arginine at position 23. Other natural and synthetic GM-CSFs, and derivatives thereof having the biological activity of natural human GM-CSF, may be equally useful in the practice of the disclosure.

In embodiments, the present recombinant human GM-CSF molecules are glycosylated. In embodiments, the present recombinant human GM-CSF molecules comprise one or more substitutions and/or deletions that impact the glycosylation of the GM-CSF.

Without wishing to be bound by theory, the degree of glycosylation of biosynthetic GM-CSFs appears to influence half-life, distribution, and elimination. (Lieschke and Burgess, N. Engl. J. Med. 327:28-35, 1992; Dorr, R. T., Clin. Ther. 15:19-29, 1993; Horgaard et al., Eur. J. Hematol. 50:32-36, 1993).

In an aspect, there is provided a recombinant human GM-CSF protein, comprising an amino acid sequence having at least about 90%, or at least about 91%, or at least about 92%, or at least about 93%, or at least about 94%, or at least about 95%, or at least about 96%, or at least about 97%, or at least about 98%, or at least about 99% identity, or 100% identity with SEQ ID NO: 1 or SEQ ID NO: 2. In an aspect, there is provided a recombinant human GM-CSF protein, comprising an amino acid sequence having at least about 90%, or at least about 91%, or at least about 92%, or at least about 93%, or at least about 94%, or at least about 95%, or at least about 96%, or at least about 97%, or at least about 98%, or at least about 99% identity, or 100% identity with SEQ ID NO: 1 or SEQ ID NO: 2 and having a substitution or deletion at position N37 or a position corresponding thereto.

In embodiments, the amino acid at position N37 or a position corresponding thereto of the recombinant human GM-CSF is a polar and neutral of charge hydrophilic amino acid. In embodiments, the polar and neutral of charge hydrophilic amino acid is selected from glutamine (Q), serine (S), threonine (T), proline (P), and cysteine (C). In embodiments, the polar and neutral of charge hydrophilic amino acid is glutamine (Q). In embodiments, the polar and neutral of charge hydrophilic amino acid is selected from glutamine (Q), serine (S), and threonine (T).

In embodiments, there is provided a recombinant human GM-CSF protein, comprising an amino acid sequence having at least about 90%, or at least about 91%, or at least about 92%, or at least about 93%, or at least about 94%, or at least about 95%, or at least about 96%, or at least about 97%, or at least about 98%, or at least about 99% identity, or 100% identity with SEQ ID NO: 1 or SEQ ID NO: 2 and having one or more of N37 deletion, N37Q, N37S, N37T, N37P, and N37C.

In embodiments, the recombinant human GM-CSF has an amino acid sequence of SEQ ID NO: 1 or SEQ ID NO: 2, or a variant of about 90%, or about 93%, or about 95%, or about 97%, or about 98% identity thereto, optionally with the present substitutions and/or deletions.

In embodiments, the GM-CSF is one of molgramostim, sargramostim, and regramostim, optionally having the present substitutions and/or deletions.

Without wishing to be bound by theory, the core of hGM-CSF consists of four helices that pack at angles. Crystal structures and mutagenic analysis of rhGM-CSF (Rozwarski D A et al., Proteins 26:304-13, 1996) showed that, in addition to apolar side chains in the protein core, 10 buried hydrogen bonding residues involve intramolecular hydrogen bonding to main chain atoms that were better conserved than residues hydrogen bonding to other side chain atoms; 24 solvation sites were observed at equivalent positions in the two molecules in the asymmetric unit, and the strongest among these was located in clefts between secondary structural elements. Two surface clusters of hydrophobic side chains are located near the expected receptor binding regions. Mutagenesis of residues on the helix A/helix C face confirmed the importance of certain Glu, Gly, and Gln residues. These residues are therefore not to be substituted in the functional substitution variants of hGM-CSF for use in the present disclosure and these helices are to be retained in a functional fragments or deletion variants of hGM-CSF for use in this disclosure.

Further, in embodiments, one of ordinary skill can reference UniProtKB entry P04141 for structure information to inform the identity of variants.

The N-terminal helix of hGM-CSF governs high affinity binding to its receptor (Shanafelt A B et al., EMBO J 10:4105-12, 1991) Transduction of the biological effects of GM-CSF requires interaction with at least two cell surface receptor components, (one of which is shared with the cytokine IL-5). The above study identified receptor binding determinants in GM-CSF by locating unique receptor binding domains on a series of human-mouse hybrid GM-CSF cytokines. The interaction of GM-CSF with the shared subunit of their high affinity receptor complexes was governed by a very small part of the peptide chains. The presence of a few key residues in the N-terminal α-helix of was sufficient to confer specificity to the interaction.

Accordingly, in embodiments, this information may inform a skilled artisan with regard to acceptable variations in the amino acid sequences.

In embodiments, the amino acid mutations are amino acid substitutions, and may include conservative and/or non-conservative substitutions.

“Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe.

As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices.

As used herein, “non-conservative substitutions” are defined as exchanges of an amino acid by another amino acid listed in a different group of the six standard amino acid groups (1) to (6) shown above.

In various embodiments, the substitutions may also include non-classical amino acids (e.g. selenocysteine, pyrrolysine, N-formylmethionine β-alanine, GABA and δ-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β methyl amino acids, C α-methyl amino acids, N α-methyl amino acids, and amino acid analogs in general).

Modification of the amino acid sequences may be achieved using any known technique in the art e.g., site-directed mutagenesis or PCR based mutagenesis. Such techniques are described, for example, in Sambrook et al., Molecular Cloning: A Laboratory Manual, Cold Spring Harbor Press, Plainview, N.Y., 1989 and Ausubel et al., Current Protocols in Molecular Biology, John Wiley & Sons, New York, N.Y., 1989.

### Glycoforms

In embodiments, the present fusion or chimeric protein contains GM-CSF molecules comprising a plurality of molecular forms. In embodiments, the molecular forms are selected from non-glycosylated, O-glycosylated, N-glycosylated and N+O glycosylated forms.

In embodiments, the fusion or chimeric protein is substantially free of hyperglycosylated, e.g. hypermannosylated forms. In embodiments, the recombinant human GM-CSF has about, or less than about, 10% hypermannosylated forms after purification, or about, or less than about, 5% hypermannosylated forms after purification, or about, or less than about, 3% hypermannosylated forms after purification, or about, or less than about, 2% hypermannosylated forms after purification, or about, or less than about, 1% hypermannosylated forms after purification.

In embodiments, the fusion or chimeric protein has less hypermannosylated forms than wild type human GM-CSF and/or sargramostim, when expressed and purified in the same manner. In embodiments, the recombinant human GM-CSF has about 100%, or about 90%, or about 80%, or about 70%, or about 60%, or about 50%, or about 40%, or about 30%, or about 20%, or about 10% less hypermannosylated forms than wild type human GM-CSF and/or sargramostim, when expressed and purified in the same manner. In embodiments, the amount of hypermannosylated forms can be detected as known in the art, e.g., without limitation, chromatographic methods (e.g. size resolution (e.g. via molecular weight and/or retention times in a column and/or fluorescent labeling (e.g. using 2-aminobenzoic acid (2-AA), 2-aminobenzamide (2-AB), and 2-aminopyridine (2-AP), anion exchange chromatography, and the like), mass spectrometry, SDS-PAGE/staining (e.g. gel-staining procedures based on the periodic acid-Schiff (PAS) reaction), affinity-based methods, such as the use of saccharide-binding proteins (e.g. lectins), enzyme-based methods, antibody-based methods, release assays (e.g. enzymatic cleavage or chemical removal of glycans or chemical derivatization), capillary electrophoresis, and eastern blot.

In embodiments, the present fusion or chimeric protein is suitable for purification that is less complicated than what is used for sargramostim.

In embodiments, the present fusion or chimeric GM-CSF proteins resolves as three peaks when quantified by reversed phase high-performance liquid chromatography (RP-HPLC). In embodiments, the present composition resolves, e.g., via HPLC, as three forms (aglycosylated form, O-linked glycoform, and O+N linked glycoform) and lack a hyperglycosylated form.

In embodiments, the present fusion or chimeric GM-CSF proteins are purifiable without a hyperglycosylated species, without the need for one or more organic solvents (e.g. without limitation, acetonitrile, trifluoroacetic acid (TFA), pyridine, acetic acid and/or N-propanol) and/or a reversed phase C4 HPLC column used for the purposes of purification and/or removal of the hyperglycosylated peak (aglycosylated form, O-linked glycoform, and O+N linked glycoform).

In embodiments, the present recombinant human GM-CSF comprises more than one species (e.g. glycoforms).

In embodiments, the fusion or chimeric protein produced in a mammalian cell, such as a Chinese Hamster Ovary (CHO) cell is monomeric with heterogenous glycoforms with both glycosylated and aglycosylated isoforms.

### Humanized Single Domain Antibody (VHH)

In aspects, the humanized single domain antibody of the present composition comprises an amino acid sequence having at least about 70%, or at least about 75%, or at least about 80%, or at least about 85%, or at least about 90%, or at least about 95%, or at least about 98%, or at least about 99% identity with SEQ ID NO: 3 and having substitutions or deletions within a non-human llama antibody framework to enable humanization of the non-human antibody. The CDRs are defined as IMGT nomenclature. CDRs can also be defined by Kabat nomenclature.

In aspects, the humanized single domain antibody of the present composition comprises an amino acid sequence having at least about 80%, or at least about 85%, or at least about 90%, or at least about 95% identity with SEQ ID NO. 3 and/or comprises three CDRs selected from (i) SEQ ID NOs: 12-14, or variants thereof or (ii) SEQ ID NOs: 15-17, or variants thereof.

In embodiments, the humanized single domain antibody (VHH) of the present composition binds human, mouse and rhesus serum albumin. In embodiments, the humanized single domain antibody (VHH) of the present composition binds and/or is specific for human serum albumin (HSA). In embodiments, the composition of the present disclosure binds HSA wherein the HSA is present in human blood plasma.

In embodiments, the present composition does not substantially compete with HSA binding to neonatal Fc receptor (FcRn).

In embodiments, the present composition binds protein A.

In embodiments, when the present fusion or chimeric protein is soluble, the protein-binding activity can be presented in terms of the dissociation constant (KD). Optionally, when the fusion or chimeric protein is a membrane antigen, the antigen-binding activity can be presented in terms of the apparent dissociation constant (apparent KD).

In embodiments, the dissociation constant, KD, and apparent dissociation constant (apparent KD) can be determined by methods known to those skilled in the art, for example, using Biacore (GE healthcare), Scatchard plot, or FACS.

In embodiments, the present disclosure provides composition having a KD of at least about 50 nM, or at least about 45 nM, or at least about 40 nM, or at least about 35 nM, or at least about 30 nM, or at least about 25 nM, or at least about 20 nM, or at least about 15 nM, or at least about 10 nM, or at least about 5 nM. In embodiments, the present disclosure provides composition having an apparent KD of at least about 100 pM, or at least about 90 pM, or at least about 80 pM, or at least about 70 pM, or at least about 60 pM, or at least about 50 pM, or at least about 40 pM, or at least about 30 pM, or at least about 20 pM or at least about 10 pM.

In embodiments, the binding activity of the composition may be determined at different conditions. In a preferred embodiment, measurement conditions are constant.

### Linker

In aspects, the fusion or chimeric protein, the protein comprising a recombinant human GM-CSF protein is connected and/or fused to a humanized single domain antibody via a linker.

In embodiments, the linker of the present composition is a flexible linker. In embodiments, the flexible linker of the present composition is substantially comprised of glycine and serine residues. In embodiments, the flexible linker of the present composition comprises at least about 20, or at least about 30, or at least about 40, or at least about 50, or at least about 60 amino acid residues.

In an embodiment, the flexible linker of the present composition comprises (Gly₄Ser)ₙ, where n is from about 1 to about 12.

In an embodiment, the flexible linker of the present composition comprises (Gly₄Ala)ₙ, where n is from about 1 to about 12.

In an embodiment, the flexible linker of the present composition is a hinge-CH2-CH3 Fc domain. In embodiment, the hinge-CH2-CH3 Fc domain of the present composition is derived from a human IgG. In embodiments, the hinge-CH2-CH3 Fc domain of the present composition is derived from human IgG1, IgG2 or IgG4.

In embodiments, the linker of the present composition comprises an amino acid sequence selected from SEQ ID NO: 4 or SEQ ID NO: 5 or SEQ ID NO: 6. In embodiments, the linker of the present composition comprises an amino acid sequence having at least about 80%, or at least about 85%, or at least about 90%, or at least about 95% identity with SEQ ID NO: 6. In embodiments, the linker of the present composition comprises an amino acid sequence having or SEQ ID NO: 4 or SEQ ID NO: 5, or a variant thereof (e.g., having about 5, or about 4, or about 3, or about 2, or about 1 substitutions or deletions).

In aspects, the VHH domain of the present composition is positioned at the N-terminus of the fusion or chimeric protein.

In aspects, the recombinant GM-CSF of the present composition is positioned at the N-terminus of the fusion or chimeric protein.

In embodiments, the recombinant GM-CSF of the present composition comprising the N-terminus of the fusion or chimeric protein produces an increased yield as compared to a composition comprising recombinant GM-CSF at the C-terminus.

### Functional Properties of the Recombinant GM-CSF Containing Fusion or Chimeric Protein

In embodiments, the present fusion or chimeric protein, optionally having the present substitutions and/or deletions, is functionally similar to wild type human GM-CSF and/or sargramostim (e.g. differ in one or more functional parameter by no more than about 50%, or by no more than about 40%, or by no more than about 30%, or by no more than about 20%, or by no more than about 10%, or by no more than about 5%, or no more than about 5-fold, or no more than about 4-fold, or no more than about 3-fold, or no more than about 2-fold of the assayed functional parameter). In embodiments, the functional parameters of GM-CSF can be detected by assays known in the art, e.g., without limitation, proliferation assays using cells such as TF-1 cell lines, primary bone marrow cells, biochemical assays such as ILITE (EAGLE) GM-CSF (luciferase under the control of GM-CSF promoter), cell survival assays e.g. myeloid cell survival assay, cell differentiation assays and co-culture experiments.

In embodiments, the present fusion or chimeric protein, optionally with the present substitutions and/or deletions, bind and/or activate the granulocyte-macrophage colony stimulating factor receptor (GM-CSF-R-alpha or CSF2R). In embodiments, the present recombinant human GM-CSF molecules, optionally with the present substitutions and/or deletions, bind and/or activate the granulocyte-macrophage colony stimulating factor receptor (GM-CSF-R-alpha or CSF2R) at an affinity, efficacy, and/or bioactivity that is comparable to wild type human GM-CSF and/or sargramostim (e.g. differ in one or more functional parameter by no more than about 50%, or by no more than about 40%, or by no more than about 30%, or by no more than about 20%, or by no more than about 10%, or by no more than about 5%, or no more than about 5-fold, or no more than about 4-fold, or no more than about 3-fold, or no more than about 2-fold). Assays for GM-CSF binding and activation are known in the art. Non-limiting examples of such assays include, for example, radioligand assays or non-radioligand assays (e.g. immunoprecipitation (IP), enzyme-linked immunosorbent assay (ELISA), western blot, fluorescence polarization (FP). fluorescence resonance energy transfer (FRET), surface plasmon resonance (SPR), and radioimmunoassay (RIA). The binding kinetics also can be assessed by standard assays known in the art, such as by Biacore analysis. Whole cell ligand-binding assays, and cell-free assay systems using soluble GM-CSF receptor alpha (sGMRa) may also be used. Some other types of assays that may be used include, receptor-binding, or saturation binding, or competitive binding assays using radio-iodinated GM-CSF, as well as cell proliferation assays.

In embodiments, the present fusion or chimeric protein can be assayed using one or more cell-based activity bioassays, e.g. using a GM-CSF dependent human cell-line proliferation assay, e.g. using TF-1, M-07e, HU-3, M-MOK, MB-02, GM/SO, F-36P, GF-D8, ELF-153, AML-193, MUTZ-3, OCI-AML5, OCI-AML6, OCI-AML1, SKNO-1, UCSD-AML1 and UT-7.

In embodiments, the potency of the present fusion or chimeric protein is measured using a bioassay employing TF-1 cells, a human erythroid leukemia cell line that proliferates in response to GM-CSF. The details of this assay are known in the art. For instance, a reference standard, control and test samples are serially diluted in triplicate in assay media and added to three separate 96-well plates. TF-1 cells in suspension are then added and the mixture is incubated at 37° C. for about 69.5-72 hours. Following the addition of a fluorescent dye (e.g. ALAMARBLUE), the plates are incubated at 37° C. for about 6.6-8 hours. TF-1 cell proliferation is then measured in a fluorescent microplate reader.

In embodiments, the present fusion or chimeric protein have roughly the specific same activity as a recombinant human GM-CSF lacking the mutations (e.g. as assayed using a bioassay employing TF-1 cells).

In embodiments, the functionality of the present fusion or chimeric protein produced in a mammalian cell, such as a Chinese Hamster Ovary (CHO) cell is comparable to a recombinant human GM-CSF produced in a yeast cell (e.g. differ in one or more functional parameter by no more than about 90%, or by no more than about 80%, or by no more than about 70%, or by no more than about 60%, or by no more than about 50%, or by no more than about 40%, or by no more than about 30%, or by no more than about 20%, or by no more than about 10%, or by no more than about 5%, or no more than about 10-fold, or no more than about 9-fold, or no more than about 8-fold, or no more than about 7-fold, or no more than about 6-fold, or no more than about 5-fold, or no more than about 4-fold, or no more than about 3-fold, or no more than about 2-fold).

In embodiments, the present fusion or chimeric protein has improved pharmacokinetics and pharmacodynamics as compared to sargramostim or WT GM-CSF. In embodiments, the present fusion or chimeric protein has an increased half-life as compared to sargramostim or WT GM-CSF (e.g. differ in one or more functional parameter by no more than about 50%, or by no more than about 40%, or by no more than about 30%, or by no more than about 20%, or by no more than about 10%, or by no more than about 5%, or no more than about 5-fold, or no more than about 4-fold, or no more than about 3-fold, or no more than about 2-fold).

In embodiments, the GM-CSF-R-alpha at which binding and/or activation occurs is expressed on the surface of a cell. In embodiments, the cell is a hematopoietic progenitor cell. In embodiments, the hematopoietic progenitor cell is an immune cell. In embodiments, the hematopoietic progenitor cell is irradiated.

In embodiments, the immunogenicity of the present fusion or chimeric protein, optionally having the present substitutions and/or deletions is comparable to wild type human GM-CSF and/or sargramostim (e.g. differ in one or more functional parameter by no more than about 50%, or by no more than about 40%, or by no more than about 30%, or by no more than about 20%, or by no more than about 10%, or by no more than about 5%, or no more than about 5-fold, or no more than about 4-fold, or no more than about 3-fold, or no more than about 2-fold). In embodiments, immunogenicity is assayed using methods known in the art. Non-limiting examples include detection of one or more anti-GM-CSF binding antibodies as assessed by, e.g. screening assays such as direct or indirect or bridging ELISA, electrochemiluminescence, bead-based chemiluminescence assays, radioimmunoprecipitation assay, surface plasma resonance and bio layer interferometry, as well as cell based luciferase reporter gene neutralizing antibody assay.

In aspects, the fusion or chimeric protein significantly increases the level, cell count, and/or amount of neutrophils, monocytes, eosinophils, total WBCs and platelets. In embodiments, the fusion or chimeric protein significantly increases the level, cell count, and/or amount of platelets.

In embodiments, the cell recombinant fusion or chimeric protein is soluble.

### Nucleic Acids and Host Cells

In embodiments, there is provided a nucleic acid molecule encoding the fusion or chimeric protein described herein. In embodiments, the nucleic acid molecule has a codon-optimized sequence.

In embodiments, the host cell is a yeast, mammalian, bacterial, insect, algae, or plant cell.

In embodiments, the yeast cell is a non-methylotrophic yeast cell. In embodiments, the host cell is a Saccharomyces cerevisiae cell.

In embodiments, there is provided a human host cell expressing the nucleic acid molecule described herein.

In embodiments, there is provided a non-human host cell expressing the nucleic acid molecule described herein.

In embodiments, there is provided a CHO cell expressing the nucleic acid molecule described herein.

### Pharmaceutical Compositions and Formulations

In embodiments, there is provided a pharmaceutical composition comprising a fusion or chimeric protein described herein and a pharmaceutically acceptable excipient or carrier.

Any pharmaceutical compositions described herein can be administered to a subject as a component of a composition that comprises a pharmaceutically acceptable carrier or vehicle. Such compositions can optionally comprise a suitable amount of a pharmaceutically acceptable excipient so as to provide the form for proper administration.

In various embodiments, pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In one embodiment, the pharmaceutically acceptable excipients are sterile when administered to a subject. Water is a useful excipient when any agent described herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents. Other examples of suitable pharmaceutical excipients are described in Remington's Pharmaceutical Sciences 1447-1676 (Alfonso R. Gennaro eds., 19th ed. 1995), incorporated herein by reference.

The present disclosure includes the described pharmaceutical compositions (and/or additional therapeutic agents) in various formulations. Any inventive pharmaceutical composition (and/or additional therapeutic agents) described herein can take the form of solutions, suspensions, emulsion, drops, tablets, pills, pellets, capsules, capsules containing liquids, gelatin capsules, powders, sustained-release formulations, suppositories, emulsions, aerosols, sprays, suspensions, lyophilized powder, frozen suspension, desiccated powder, or any other form suitable for use. In one embodiment, the composition is in the form of a capsule. In another embodiment, the composition is in the form of a tablet. In yet another embodiment, the pharmaceutical composition is formulated in the form of a soft-gel capsule. In a further embodiment, the pharmaceutical composition is formulated in the form of a gelatin capsule. In yet another embodiment, the pharmaceutical composition is formulated as a liquid.

Where necessary, the present pharmaceutical compositions (and/or additional therapeutic agents) can also include a solubilizing agent. Also, the agents can be delivered with a suitable vehicle or delivery device as known in the art. Combination therapies outlined herein can be co-delivered in a single delivery vehicle or delivery device.

The formulations comprising the inventive pharmaceutical compositions (and/or additional therapeutic agents) of the present disclosure may conveniently be presented in unit dosage forms and may be prepared by any of the methods well known in the art of pharmacy. Such methods generally include the step of bringing the therapeutic agents into association with a carrier, which constitutes one or more accessory ingredients. Typically, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In various embodiments, any pharmaceutical compositions (and/or additional therapeutic agents) described herein is formulated in accordance with routine procedures as a composition adapted for a mode of administration described herein.

Routes of administration include, for example: oral, intradermal, intramuscular, intraperitoneal, intravenous, subcutaneous, intranasal, epidural, sublingual, intranasal, intracerebral, intravaginal, transdermal, rectally, by inhalation, or topically. Administration can be local or systemic. In embodiments, the administering is effected orally. In another embodiment, the administration is by parenteral injection. The mode of administration can be left to the discretion of the practitioner, and depends in-part upon the site of the medical condition. In most instances, administration results in the release of any agent described herein into the bloodstream.

In specific embodiments, the fusion or chimeric protein, or pharmaceutical composition thereof, (and/or additional therapeutic agents) is administered via an intravenous route.

In one embodiment, the pharmaceutical compositions (and/or additional therapeutic agents) described herein are formulated in accordance with routine procedures as a composition adapted for oral administration. Compositions for oral delivery can be in the form of tablets, lozenges, aqueous or oily suspensions, granules, powders, emulsions, capsules, syrups, or elixirs, for example. Orally administered compositions can comprise one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving any pharmaceutical compositions (and/or additional therapeutic agents) described herein are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be useful. Oral compositions can include standard excipients such as mannitol, lactose, starch, magnesium stearate, sodium saccharin, cellulose, and magnesium carbonate. In one embodiment, the excipients are of pharmaceutical grade. Suspensions, in addition to the active compounds, may contain suspending agents such as, for example, ethoxylated isostearyl alcohols, polyoxyethylene sorbitol and sorbitan esters, microcrystalline cellulose, aluminum metahydroxide, bentonite, agar-agar, tragacanth, etc., and mixtures thereof.

Dosage forms suitable for parenteral administration (e.g. intravenous, intramuscular, intraperitoneal, subcutaneous and intra-articular injection and infusion) include, for example, solutions, suspensions, dispersions, emulsions, and the like. They may also be manufactured in the form of sterile solid compositions (e.g. lyophilized composition), which can be dissolved or suspended in sterile injectable medium immediately before use. They may contain, for example, suspending or dispersing agents known in the art. Formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor ELTM (BASF, Parsippany, NJ) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

Any inventive pharmaceutical compositions (and/or additional therapeutic agents) described herein can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropyl cellulose, hydropropylmethyl cellulose, polyvinylpyrrolidone, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the agents described herein. The disclosure thus provides single unit dosage forms suitable for oral administration such as, but not limited to, tablets, capsules, gelcaps, and caplets that are adapted for controlled- or sustained-release.

Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In another embodiment, a controlled-release system can be placed in proximity of the target area to be treated, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

Pharmaceutical formulations preferably are sterile. Sterilization can be accomplished, for example, by filtration through sterile filtration membranes. Where the composition is lyophilized, filter sterilization can be conducted prior to or following lyophilization and reconstitution.

### Pharmaceutically Acceptable Salts and Excipients

The composition described herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety.

Pharmaceutically acceptable salts include, byway of non-limiting example, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, pamoate, phenylacetate, trifluoroacetate, acrylate, chlorobenzoate, dinitrobenzoate, hydroxybenzoate, methoxybenzoate, methylbenzoate, o-acetoxybenzoate, naphthalene-2-benzoate, isobutyrate, phenylbutyrate, α-hydroxybutyrate, butyne-1,4-dicarboxylate, hexyne-1,4-dicarboxylate, caprate, caprylate, cinnamate, glycollate, heptanoate, hippurate, malate, hydroxymaleate, malonate, mandelate, mesylate, nicotinate, phthalate, teraphthalate, propiolate, propionate, phenylpropionate, sebacate, suberate, p-bromobenzenesulfonate, chlorobenzenesulfonate, ethylsulfonate, 2-hydroxyethylsulfonate, methylsulfonate, naphthalene-1-sulfonate, naphthalene-2-sulfonate, naphthalene-1,5-sulfonate, xylenesulfonate, and tartarate salts.

The term “pharmaceutically acceptable salt” also refers to a salt of the compositions of the present disclosure having an acidic functional group, such as a carboxylic acid functional group, and a base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-; bis-, or tris-(2-hydroxyethyl)amine, 2-hydroxy-tert-butylamine, or tris-(hydroxymethyl)methylamine, N,N-di-lower alkyl-N-(hydroxyl-lower alkyl)-amines, such as N,N-dimethyl-N-(2-hydroxyethyl)amine or tri-(2-hydroxyethyl)amine; N-methyl-D-glucamine; and amino acids such as arginine, lysine, and the like.

In embodiments, the compositions described herein are in the form of a pharmaceutically acceptable salt.

### Methods of Use

In an aspect, there is provided a method of treating a patient or subject who is undertaking or has undertaken a cancer therapy, or who is undertaking or has undertaken a bone marrow transplant, and/or who had been acutely exposed to myelosuppressive doses of radiation (e.g. hematopoietic syndrome of Acute Radiation Syndrome (H-ARS)) with or without other insults such as a burn, wound, infection, hemorrhage or blunt trauma (e.g. Radiation Combined Injury (RCI)); the method comprising administering to the patient a therapeutically effective amount of the present fusion or chimeric protein or a pharmaceutical composition thereof. In embodiments, the patient is treated by modulating clonal expansion, survival, differentiation and activation state of hematopoietic progenitor cells. In embodiments, the patient is treated by modulating a myelomonocytic cell lineage, by promoting the proliferation of megakaryocytic and erythroid progenitors. In embodiments, the patient is treated by modulating hematopoietic progenitor cells, by stimulating the survival, proliferation and activation of neutrophils, macrophages and/or dendritic cells. In embodiments, the patient is treated following bone marrow transplant by modulating hematopoietic progenitor cells, by stimulating the survival, proliferation and activation of neutrophils, macrophages and/or dendritic cells.

In an aspect, there is provided a therapeutic method comprising administering to a patient a therapeutically effective amount of the present fusion or chimeric protein or a pharmaceutical composition thereof or contacting cells with an effective amount of the pharmaceutical composition described herein and administering therapeutically effective amount of the cells, wherein the therapy: accelerates neutrophil recovery and/or to reduce the incidence of infections following induction chemotherapy; mobilizes hematopoietic progenitor cells into peripheral blood for collection by leukapheresis and transplantation; accelerates of myeloid reconstitution following autologous or allogeneic bone marrow or peripheral blood progenitor cell transplantation; treats delayed neutrophil recovery or graft failure after autologous or allogeneic bone marrow transplantation; and/or treats Hematopoietic syndrome of Acute Radiation Syndrome (H-ARS); and/or treats Radiation Combined Injury (RCI).

In an aspect, there is provided a method for treating an infection with a virus, comprising: administering an effective amount of a composition comprising the present fusion or chimeric protein or a pharmaceutical composition comprising the same to a patient in need thereof.

In embodiments, the viral infection is an influenza infection, optionally selected from Type A, Type B, Type C, and Type D influenza virus infection.

In embodiments, the viral infection is Viral Hemorrhagic Fevers, caused by a group of viruses optionally selected from Arenaviruses, Flaviviruses, Filoviruses, Hantaviruses, Nairoviruses, Phenuviruses.

In embodiments, the lung infection is Pneumonic Plague caused by an infection with Yersinia pestis.

In embodiments, the viral infection is a coronavirus infection. In embodiments, the coronavirus is a betacoronavirus, optionally selected from severe acute respiratory syndrome coronavirus 2 (SARS-CoV-2), SARS-CoV, Middle East respiratory syndrome-corona virus (MERS-CoV), HCoV-HKU1, and HCoV-OC43. In embodiments, the coronavirus is an alphacoronavirus, optionally selected from HCoV-NL63 and HCoV-229E.

The coronavirus is a member of the family Coronaviridae, including betacoronavirus and alphacoronavirus respiratory pathogens that have relatively recently become known to invade humans. The Coronaviridae family includes such betacoronavirus as Severe acute respiratory syndrome coronavirus 2 (SARS-CoV-2), SARS-CoV, Middle East Respiratory Syndrome-Corona Virus (MERS-CoV), HCoV-HKU1, and HCoV-OC43. Alphacoronavirus includes, e.g., HCoV-NL63 and HCoV-229E.

Coronaviruses invade cells through “spike” surface glycoprotein that is responsible for viral recognition of Angiotensin Converting Enzyme 2 (ACE2), a transmembrane receptor on mammalian hosts that facilitate viral entrance into host cells. Zhou et al., A pneumonia outbreak associated with a new coronavirus of probable bat origin. Nature 2020. A new coronavirus infection 2019 (COVID-19) is caused by SARS-CoV-2.

SARS-CoV-2 is a new virus thought to be originated from the bat. COVID-19 causes severe respiratory distress and this RNA virus strain has been the cause of the recent outbreak that has been declared a major threat to public health and worldwide emergency. Phylogenetic analysis of the complete genome of SARS-CoV-2 revealed that the virus was most closely related (89.1% nucleotide similarity) to a group of SARS-like coronaviruses (genus Betacoronavirus, subgenus Sarbecovirus). Wu et al., A new coronavirus associated with human respiratory disease in China. Nature, Feb. 3, 2020.

The SARS-CoV-2 is an enveloped, single stranded, RNA virus that encodes a “spike” protein, also known as the S protein, which is a surface glycoprotein that mediates binding to a cell surface receptor; an integral membrane protein; an envelope protein, and a nucleocapsid protein. The S protein, comprising S1 subunit and S2 subunit, is a trimeric class I fusion protein that exists in a prefusion conformation that undergoes a structural rearrangement to fuse the viral membrane with the host-cell membrane. See, e.g., Li, F. Structure, Function, and Evolution of Coronavirus Spike Proteins. Annu. Rev. Virol. 3: 237-261(2016), which is incorporated herein by reference in its entirety. The structure of the SARS-CoV-2 spike protein in the prefusion conformation has been discovered. See Daniel et al., Cryo-EM structure of the SARS-CoV-2 spike in the prefusion conformation. Science, 19 Feb. 2020, which is incorporated herein by reference in its entirety.

Phylogenetic analysis of the complete genome of SARS-CoV-2 (GenBank Accession No.: MN908947) revealed that the virus was most closely related (89.1% nucleotide similarity) to a group of SARS-like coronaviruses (genus Betacoronavirus, subgenus Sarbecovirus). Wu et al., A new coronavirus associated with human respiratory disease in China. Nature, Feb. 3, 2020, which is incorporated herein by reference in its entirety.

The SARS-CoV-2 has a spike surface glycoprotein, membrane glycoprotein M, envelope protein E, and nucleocapsid phosphoprotein N. The complete genome of the SARS-CoV-2 coronavirus (29903 nucleotides, single-stranded RNA) is described in the NCBI database as GenBank Reference Sequence: MN908947. The coronavirus protein can be selected from: coronavirus spike protein (GenBank Reference Sequence: QHD43416), coronavirus membrane glycoprotein M (GenBank Reference Sequence: QHD43419), coronavirus envelope protein E (GenBank Reference Sequence: QHD43418), and coronavirus nucleocapsid phosphoprotein E (GenBank Reference Sequence: QHD43423).

In embodiments, the method prevents or mitigates development of acute respiratory distress syndrome (ARDS) in the patient.

In embodiments, the coronavirus is SARS-CoV-2. In embodiments, the patient is afflicted with COVID-19. In embodiments, the patient is afflicted with one or more of fever, cough, shortness of breath, diarrhea, upper respiratory symptoms, lower respiratory symptoms, pneumonia, and acute respiratory syndrome.

In embodiments, the patient is hypoxic. In embodiments, the patient is afflicted with respiratory distress. In embodiments, the method improves oxygenation in the patient. In embodiments, the method prevents or mitigates a transition from respiratory distress to cytokine imbalance in the patient. In embodiments, the method reverses or prevents a cytokine storm. In embodiments, the method reverses or prevents a cytokine storm in the lungs or systemically. In embodiments, the cytokine storm is selected from one or more of systemic inflammatory response syndrome, cytokine release syndrome, macrophage activation syndrome, and hemophagocytic lymphohistiocytosis. In embodiments, the method reverses or prevents excessive production of one or more inflammatory cytokines. In embodiments, the inflammatory cytokine is one or more of IL-6, IL-1, IL-1 receptor antagonist (IL-1ra), IL-2ra, IL-10, IL-18, TNFα, interferon-γ, CXCL10, and CCL7.

In embodiments, the method causes a decrease in viral or bacterial load in the patient relative to before treatment.

In embodiments, the viral infection is selected from a betacoronavirus infection, optionally selected from severe acute respiratory syndrome coronavirus 2 (SARS-CoV-2), severe acute respiratory syndrome coronavirus (SARS-CoV-1), Middle East Respiratory Syndrome-Corona Virus (MERS-CoV), HCoV-HKU1, and HCoV-OC43 infection. In embodiments, the viral infection is selected from an alphacoronavirus infection, optionally selected from HCoV-NL63 and HCoV-229E infection.

In embodiments, the betacoronavirus infection is severe acute respiratory syndrome (SARS).

In embodiments, the betacoronavirus infection is, or is associated with, coronavirus disease 2019 (COVID-19).

In embodiments, the viral infection is an influenza infection, optionally selected from Type A, Type B, Type C, and Type D influenza virus infection. In embodiments, the influenza infection is pandemic 2009 influenza A (H1N1) or avian influenza A (H5N1).

In embodiments, the viral infection is Viral Hemorrhagic Fevers, caused by a group of viruses optionally selected from Arenaviruses, Flaviviruses, Filoviruses, Hantaviruses, Nairoviruses, Phenuviruses.

In embodiments, the lung infection is Pneumonic Plague caused by an infection with Yersinia pestis.

In an aspect, there is provided a method for treating symptoms caused by exposure to chemical warfare agents, comprising: administering an effective amount of a composition comprising the present fusion or chimeric protein or a pharmaceutical composition comprising the same to a patient in need thereof.

In embodiments, the chemical warfare agent is Sulphur Mustard gas (SM/HD) and/or chlorine gas.

In embodiments, the symptom of exposure in the patient is characterized by myelosuppression.

In embodiments, the method provided causes a reduction and/or amelioration of symptoms, including myelosuppression, caused by exposure to Sulphur Mustard gas (SM/HD) and/or chlorine gas.

In an aspect, there is provided a method of treating and/or ameliorating symptoms caused by a neurodegenerative disease in a patient or subject; the method comprising administering to the patient a therapeutically effective amount of the present fusion or chimeric protein or a pharmaceutical composition thereof.

In embodiments, the neurodegenerative disease is selected from Alzheimer's disease, Parkinson's disease, progressive supranuclear palsy (PSP), multiple system atrophy (MSA), Lewy body dementia, Parkinson's disease dementia epilepsy, stroke, Huntington's Chorea, cerebral hypoxia, multiple sclerosis, amyotrophic lateral sclerosis (ALS), and peripheral neuropathy.

In embodiments, the neurodegenerative disease in the patient is characterized by oxidative stress, loss of neurite integrity, apoptosis, neuronal loss or/and inflammation response. In embodiments, the neurodegenerative disease is associated with cognitive impairment.

In an aspect, there is provided a therapeutic method comprising administering to a patient with a neurodegenerative disease a therapeutically effective amount of the present fusion or chimeric protein or a pharmaceutical composition thereof or contacting cells with an effective amount of the pharmaceutical composition described herein and administering therapeutically effective amount of the cells, wherein the therapy: elicits a disease-modifying response; temporarily or permanently slows down cognitive decline; causes an amelioration of symptoms; and/or slows the onset and/or development of the disease.

In an aspect, the present fusion or chimeric protein provided is a composition that promotes the enhanced delivery of recombinant GM-CSF through the endothelial capillaries (blood brain barrier, BBB) for central nervous system (CNS) therapeutics.

In an aspect, there is provided a method of treating and/or ameliorating symptoms caused by an autoimmune disease in a patient or subject; the method comprising administering to the patient a therapeutically effective amount of the present fusion or chimeric protein or a pharmaceutical composition thereof.

In an embodiment, the autoimmune disease is optionally selected from Crohn's disease and/or ulcerative colitis (UC) and/or irritable bowel disease (IBD).

In an aspect, there is provided a therapeutic method comprising administering to a patient with an autoimmune disease a therapeutically effective amount of the present fusion or chimeric protein or a pharmaceutical composition thereof or contacting cells with an effective amount of the pharmaceutical composition described herein and administering therapeutically effective amount of the cells, wherein the therapy can restore the balance of effector and regulatory immune function.

In an aspect, there is provided a method of treating and/or ameliorating symptoms in a patient or subject caused by a caspase recruitment domain family member 9 (CARD9) deficiency.

In an aspect, there is provided a method of treating a patient or subject who is undertaking or has undertaken a therapy for fungal infections; the method comprising administering to the patient a therapeutically effective amount of the present fusion or chimeric protein or a pharmaceutical composition thereof.

In embodiments, the present composition is used as an adjunctive therapy to anti-fungal therapies. In embodiments, the present composition decreases and/or reduces fungal load in various tissues.

In an aspect, there is provided a method of treating a patient or subject who is undertaking or has undertaken a therapy for hematopoietic syndrome of Acute Radiation Syndrome (H-ARS).

In an aspect, there is provided a method of treating a patient or subject who is undertaking or has undertaken a therapy for Radiation Combined Injury (RCI).

In an aspect, there is provided a method of treating a patient or subject who is undertaking or has undertaken a therapy for nerve transection.

In an aspect, there is provided a method of treating a patient or subject who is undertaking or has undertaken a therapy for traumatic brain injury.

In embodiments, the present composition is used as an adjunctive therapy to anti-fungal therapies. In embodiments, the present composition decreases and/or reduces fungal load in various tissues.

In an aspect, there is provided a method of treating a patient or subject who is undertaking or has undertaken a therapy for sepsis. In embodiments, the present composition balances and/or regulates inflammation and acute phase response, and to reduce morbidity and mortality rates.

In an aspect, there is provided a method of treating a patient or subject who is undertaking or who has undertaken treatment for multiple organ dysfunction syndrome (MODS).

In an aspect, there is provided a method of treating a patient or subject who is undertaking or has undertaken treatment for peripheral artery disease/peripheral arterial disease.

In an aspect, there is provided a method of treating a patient or subject who is undertaking or has undertaken treatment for chronic wound healing.

In an embodiment, the chronic wound is optionally selected lepra, leg ulcers, and/or indolent wounds of various other causes.

In embodiments, the present composition is used as an adjunctive therapy to wound healing therapies, including but not limited to hyperbaric oxygen therapy, ultrasound and electromagnetic therapy and/or negative pressure wound therapy. In embodiments, the present composition acts upon various epidermal cells and enhances wound healing. In embodiments the present composition promotes tissue remodeling and regeneration.

### Combination Therapy and Additional Therapeutic Agents

In various embodiments, the pharmaceutical composition of the present disclosure is co-administered in conjunction with additional agent(s). Co-administration can be simultaneous or sequential.

In one embodiment, the additional therapeutic agent and the fusion or chimeric protein, or pharmaceutical composition thereof, of the present disclosure are administered to a subject simultaneously. The term “simultaneously” as used herein, means that the additional therapeutic agent and the fusion or chimeric protein, or pharmaceutical composition thereof, are administered with a time separation of no more than about 60 minutes, such as no more than about 30 minutes, no more than about 20 minutes, no more than about 10 minutes, no more than about 5 minutes, or no more than about 1 minute. Administration of the additional therapeutic agent and the fusion or chimeric protein, or pharmaceutical composition thereof, can be by simultaneous administration of a single formulation (e.g., a formulation comprising the additional therapeutic agent and the fusion or chimeric protein, or pharmaceutical composition thereof,) or of separate formulations (e.g., a first formulation including the additional therapeutic agent and a second formulation including the fusion or chimeric protein).

Co-administration does not require the therapeutic agents to be administered simultaneously, if the timing of their administration is such that the pharmacological activities of the additional therapeutic agent and the fusion or chimeric protein, or pharmaceutical composition thereof, overlap in time, thereby exerting a combined therapeutic effect. For example, the additional therapeutic agent and the targeting moiety, the fusion or chimeric protein, or pharmaceutical composition thereof, composition can be administered sequentially. The term “sequentially” as used herein means that the additional therapeutic agent and the fusion or chimeric protein are administered with a time separation of more than about 60 minutes. For example, the time between the sequential administration of the additional therapeutic agent and the fusion or chimeric protein, or pharmaceutical composition thereof, can be more than about 60 minutes, more than about 2 hours, more than about 5 hours, more than about 10 hours, more than about 1 day, more than about 2 days, more than about 3 days, more than about 1 week apart, more than about 2 weeks apart, or more than about one month apart. The optimal administration times will depend on the rates of metabolism, excretion, and/or the pharmacodynamic activity of the additional therapeutic agent and the fusion or chimeric protein, or pharmaceutical composition thereof, being administered. Either the additional therapeutic agent or the fusion or chimeric protein composition, or pharmaceutical composition thereof, may be administered first.

Co-administration also does not require the therapeutic agents to be administered to the subject by the same route of administration. Rather, each therapeutic agent can be administered by any appropriate route, for example, parenterally or non-parenterally.

In embodiments, the fusion or chimeric protein, or pharmaceutical composition thereof, described herein acts synergistically when co-administered with another therapeutic agent. In such embodiments, the targeting moiety, the present composition and the additional therapeutic agent may be administered at doses that are lower than the doses employed when the agents are used in the context of monotherapy.

In embodiments, the additional therapeutic agent is an anti-viral drug.

In embodiments, the additional therapeutic agent is selected from drugs including antivirals such as remdesivir, favipiravir, oseltamivir, baloxavir, galidesivir, amprenavir, tipranavir, saquinavir, nelfinavir, indinavir, darunavir, atazanavir, emetine, lopinavir and/or ritonavir, arbidol and lopinavir/ritonavir, and/or ribavirin, darunavir and cobicistat, and/or IFN-beta-1b, β-D-N4-hydroxycytidine (NHC) such as EIDD-1931 or EIDD-2801 or EIDD-2801; immunomodulators such as glucocorticoids, IFN-a 2a, IFN-a 2b, IFN-b, pegylated IFN-g, baricitinib, sirolimus, clazakizumab, canakinumab, XPro1595, tocilizumab, sarilumab, siltuximab, adalimumab, eculizumab, ivermectin, anakinra, prezcobix, xiyanping, fingolimod, methylprednisolone, leronlimab, thalidomide, MK-2206, nicolasamide, nitazoxamide, chloroquine or hydroxychloroquine; antibiotics such as carrimycin, brilacidin, azithromycin, valinomycin, angiotension inhibitors/antagonists like rhACE2/GSK2586881/APN01, losartan, eprosartan, telmisartan, valsartan; serine protease inhibitor including camostat mesylate, nafamostat other drugs such as bromhexine, aprotinin, chlorpromazine, zotatifin, methotrexate, lenalidomide, anti-VEGF-A and Intravenous Immunoglobulin (IVIG). For instance, in embodiments, any of these additional therapeutic agents find use in the context of a SARS-CoV-2 infection.

In embodiments, the additional therapeutic agent is selected from favipiravir, Ianinamivir octanoate, peramivir, zanamivir, oseltamivir phosphate, baloxavir marboxil, umifenovir, urumin amantadine hydrochloride, rimantadine hydrochloride, adapromine, LASAG/BAY81-87981, celecoxib, etanercept, metformin, gemcitabine, dapivirine, trametinib, lisinopril, naproxen, nalidixic acid, dorzolamide, ruxolitinib, midodrine, diltiazem; statins including atorvastatin, nitazoxanide; PPAR antagonists including gemfibrozil. For instance, in embodiments, any of these additional therapeutic agents find use in the context of an influenza infection or other viral infections such as those that cause viral hemorrhagic fevers.

In embodiments, the additional therapeutic agent is a drug(s) to treat a neurodegenerative disease.

In embodiments, the additional therapeutic agent is selected from Levodopa, cholinesterase inhibitors including donepezil (ARICEPT), rivastigmine (EXELON), Galantamine (RAZADYNE), atypical antipsychotics/second generation antipsychotics including serotonin-dopamin antagonists (SDAs), multi-acting receptor-targeted antipsychotics (MARTAs), and D2 partial agonists (e.g. ABILIFY/Aripiprazol), NMDA receptor antagonist memantine, riluzole (RILUTEK), NSAIDs (non-steroidal anti-inflammatory drugs), caffein A2A receptor antagonists and CERE-120 (adeno-associated virus serotype 2-neurturin), and deep brain stimulation. For instance, in embodiments, any of these therapeutic agents find use in the context of a neurodegenerative disease.

In embodiments, the additional therapeutic agent is an anti-fungal drug.

In embodiments, the additional therapeutic agent is selected from nystatin; amphotericin B; terbinafine; armamentarium; pyrimidine analogues such as flucytosine; triazoles such as fluconazole, isavuconazole, itraconazole, pramiconazole, ravuconazole, voriconazole and posaconazole; imidazoles such as clotrimazole, econazole, ketoconazole and miconazole; echinocandins such as caspofungin, micafungin, and anidulafungin. For instance, in embodiments, any of these therapeutic agents find use in the context of a fungal infection.

In embodiments, the additional therapeutic agent is a drug(s) to treat an autoimmune disease.

In embodiments, the additional therapeutic agent is selected from corticosteroids such as prednisone and budesonide (Entocort EC); oral 5-aminosalicylates such as sulfasalazine (AZULFIDINE); anti-inflammatory drugs/inflammatory suppressors/immunosuppressants such as azathioprine (AZASAN, IMURAN), mercaptopurine (PURINETHOL, PURIXAN), methotrexate (TREXALL) and corticosteroids and aminosalicylates like mesalamine (ASACOL HD, DELZICOL), balsalazide (COLAZAL), tofacitinib (XELJANZ), cyclosporine (GENGRAF, NEORAL, SANDIMMUNE) and olsalazine (DIPENTUM); biologies such as natalizumab (TYSABRI), vedolizumab (ENTYVIO), infliximab (REMICADE), adalimumab (HUMIRA), certolizumab pegol (CIMZIA), ustekinumab (STELARA) and golimumab (SIMPONI); antibiotics such as ciprofloxacin (CIPRO) and metronidazole (FLAGYL); anti-diarrheals such as loperamide (IMODIUM A-D); pain relievers; vitamins and supplements including iron supplements. For instance, in embodiments, any of these therapeutic agents find use in the context of an autoimmune disease.

In embodiments, the additional therapeutic agent is a drug(s) to accelerate or help in wound healing or other dermatological conditions.

In embodiments, the additional therapeutic agent is selected from hyperbaric oxygen therapy, ultrasound and electromagnetic therapy and/or negative pressure wound therapy; corticosteroids such as hydrocortisone, triamcinolone (ACETONIDE, TRIANEX), clobetasol (TEMOVATE); vitamin D analogues such as calcipotriene and calcitriol (VECTICAL); retinoids such as tazarotene (TAZORAC, AVAGE) and acitretin (SORIATANE), calcineurin inhibitors such as tacrolimus (PROTOPIC) and pimecrolimus (ELIDEL); salicylic acid; coal tar; goeckerman therapy; anthralin; light therapies such as natural sunlight, UVB broadband, UVB narrowband, psoralen plus ultraviolet A (PUVA) and excimer laser therapy; immunosuppresants like steroids, methotrexate (TREXALL), adalimumab (HUMIRA), infliximab (REMICADE), cyclosporines; biologics etanercept (ENBREL), infliximab (REMICADE), adalimumab (HUMIRA), ustekinumab (STELARA), secukinumab (COSENTYX) and ixekizumab (TALTZ); thioguanine (TABLOID) and hydroxyurea (DROXIA, HYDREA), aloe extract cream, fish oil supplements, Oregon grape and essential oils.

In embodiments, the additional therapeutic agent is a cardiovascular disease or peripheral arterial disease drug.

In embodiments, the additional therapeutic agent is selected from anticoagulants such as apixaban (ELIQUIS), warfarin (COUMADIN); antiplatelet and dual antiplatelet therapy (DAPT) such as asprin along with P2Y12 inhibitors such as clopidogrel (PLAVIX), Prasugrel (EFFIENT), Ticagrelor (BRILINTA), Dipyridamole (PERSANTINE); angiotensin-converting enzyme (ACE) inhibitors such as benazepril (LOTENSIN); angiotensin II receptor blockers (or inhibitors) such as Azilsartan (EBARBI). angiotensin receptor-neprilysin inhibitors (ARNIs) such as sacubitril/valsartan (ENTRESTO); beta blockers (beta-adrenergic blocking agents) such as acebutolol (SECTRAL); combine alpha and beta-blockers such as carvedilol (COREG, COREG CR); calcium channel blockers such amlodipine (NORVASC); cholesterol-lowering medications such as statins (atorvastation/LIPITOR), nicotinic acids (niacin); cholesterol absorption inhibitor (ezetimibe/ZETIA), combination station and cholesterol absorption inhibitors (Ezetimibe/Simvastatin (Vytorin); digitalis (digoxin/LANOXIN); diuretics such as acetazolamide (DIAMOX); vasodialators such as isosorbide dinitrate (ISORDIL) and nitroglycerine (NITRO BID, NITRO STAT).

In embodiments the additional therapeutic agent is a drug to treat sepsis or septicemia.

In embodiments, the additional therapeutic agent is selected from antibiotics such as ceftriaxone (ROCEPHIN), cefotaxime (CLAFORAN), ampicillin and sulbactam (UNASYN), ceftazidime (FORTAZ), and clindamycin (CLEOCIN).

In embodiments, the additional therapeutic agent is a drug(s) to treat an oncology indication.

In embodiments, the additional therapeutic agent is selected from cytotoxic agent including toxins such as ricin; chemotherapeutic agents such as 5-fluorouracil, radioisotopes (e.g. iodine-131, radium-223), targeted therapies such as HERCEPTIN and immunooncology agents such as ipilimumab (YERVOY) and nivolumab (OPDIVO).

In embodiments, the additional therapy is a drug to increase platelet cell number or count and/or treat immune thrombopenia (ITP).

In embodiments, the additional therapeutic agent is selected from steroids such as prednisone, immune globulin therapy, romiplostim (NPLATE), eltrombipag (PROMACTA) and rituximab (RITUXAN, TRUXIMA).

### Methods of Making

In aspects, there is provided a method of making a fusion or chimeric protein comprising: (a) obtaining a cell transfected with a nucleic acid encoding a recombinant human GM-CSF, comprising an amino acid sequence having at least about 97% identity with SEQ ID NO: 1 or SEQ ID NO: 2, and optionally having a substitution or deletion at position N37, or a position corresponding thereto, or an extract thereof, a linker and a humanized single domain antibody; (b) purifying the fusion or chimeric protein from the transfected cell using one or more HPLC columns; and (c) collecting the purified fusion or chimeric protein, the purified chimeric protein being substantially free of hypermannosylated GM-CSF forms.

In embodiments, the cell is a prokaryotic or eukaryotic host cell, e.g. yeast, mammalian, bacterial, insect, algae, or plant cell.

Suitable prokaryotic host cells include bacterial cells (e.g., E coli, Bacillus subtilis, Mycobacterium spp., M. tuberculosis, or other appropriate bacterial cells), and archaeal cells (e.g. Methanococcus jannaschii and Methanococcus maripaludis).

In embodiments, the host cell of the present disclosure is a eukaryotic host cell. Suitable eukaryotic host cells include, but are not limited to: fungal cells, algal cells, insect cells, animal cells (e.g., mammalian cells, avian cells, and fish cells), and plant cells.

Suitable mammalian host cells include, but not limited to: Chinese hamster ovary (CHO) cells, human embryonic kidney (HEK) 293 cells.

Suitable fungal host cells include, but are not limited to: Ascomycota, Basidiomycota, Deuteromycota, Zygomycota, Fungi imperfecti.

Suitable yeast host cells include, but are not limited to: Candida, Hansenula, Saccharomyces, Schizosaccharomyces, Kluyveromyces, and Yarrowia. In embodiments, the yeast cell is Hansenula polymorpha, Saccharomyces cerevisiae, Saccaromyces carlsbergensis, Saccharomyces diastaticus, Saccharomyces norbensis, Saccharomyces kluyveri, Schizosaccharomyces pombe, Kluyveromyces lactis, Candida albicans, or Yarrowia lipolytica.

Suitable filamentous fungi host cells include, for example, any filamentous forms of the subdivision Eumycotina and Oomycota. In embodiments, the filamentous fungal host cell may be a cell of a species of: Achlya, Acremonium, Aspergillus, Aureobasidium, Bjerkandera, Ceriporiopsis, Cephalosporium, Chrysosporium, Cochliobolus, Corynascus, Cryphonectria, Cryptococcus, Coprinus, Coriolus, Diplodia, Endothis, Fusarium, Gibberella, Gliocladium, Humicola, Hypocrea, Myceliophthora (e.g., Myceliophthora thermophila), Mucor, Neurospora, Penicillium, Podospora, Phlebia, Piromyces, Pyricularia, Rhizomucor, Rhizopus, Schizophyllum, Scytalidium, Sporotrichum, Talaromyces, Thermoascus, Thielavia, Tramates, Tolypocladium, Trichoderma, Verticillium, Volvariella, or teleomorphs, or anamorphs, and synonyms or taxonomic equivalents thereof. In one embodiment, the filamentous fungus is selected from A. nidulans, A. oryzae, A. sojae, and Aspergilli of the A. niger Group. In an embodiment, the filamentous fungus is Aspergillus niger.

In aspects, there is provided a method of method of making a composition comprising a fusion or chimeric protein comprising: (a) obtaining a cell transfected with a nucleic acid encoding a recombinant human GM-CSF, comprising an amino acid sequence having at least about 97% identity with SEQ ID NO: 2 and having a substitution or deletion at position N37, or a position corresponding thereto, or an extract thereof, a linker and a VHH humanized nanobody, (b) purifying the fusion or chimeric protein from the transfected cell using one or more HPLC columns, (c) collecting the purified fusion or chimeric protein, the purified fusion or chimeric protein being substantially free of hypermannosylated GM-CSF forms.

In embodiments, the cell is a yeast cell, e.g. without limitation Saccharomyces cerevisiae.

In embodiments, the cell is a mammalian cell, e.g. without limitation Chinese Hamster Ovary (CHO) cell.

In embodiments, production of the fusion or chimeric protein in a mammalian cell increases expression levels of the present composition, as compared to production using a non-mammalian cell.

In embodiments, the method further comprises formulating the purified fusion or chimeric protein for injection, e.g. subcutaneous or intravenous injection.

### Sequences

SEQ ID NO: 1 is wild type GM-CSF. The sites of the substitutions or deletions of some embodiments are indicated by underlining and bold:

APARSPSPSTQPWEHVNAIQEARRLLNLSRDTAAEMNETVEVISE
MFDLQEPTCLQTRLELYKQGLRGSLTKLKGPLTMMASHYKQHCPP
TPETSCATQIITFESFKENLKDFLLVIPFDCWEPVQE.

SEQ ID NO: 2 is sargramostim. The sites of the substitutions or deletions of some embodiments are indicated by underlining and bold:

APARSPSPSTQPWEHVNAIQEALRLLNLSRDTAAEMNETVEVISE
MFDLQEPTCLQTRLELYKQGLRGSLTKLKGPLTMMASHYKQHCPP
TPETSCATQIITFESFKENLKDFLLVIPFDCWEPVQE.

SEQ ID NO: 3 is human VH3-23 sequence.

MEFGLSWLFLVAILKGVQCEVOLVESGGGLVQPGGSLRLSCAASG
FTFSSYAMSWVRQAPGKGLEWVSAISGSGGSTYYADSVKGRFTIS
RDNSKNTLYLQMNSLRAEDTAVYYCAK.

SEQ ID NO: 4 is G4S linker sequence.

GGGGSGGGGSGGGGSGGGGS

SEQ ID NO: 5 is G4A linker sequence.

GGGGAGGGGAGGGGAGGGGA

SEQ ID NO: 6 is a variant of a hinge sequence from human IgG1 sequence:

EPKSSDKTHTSPPSPAPE.

SEQ ID NO: 7 is VHH-GMCSF construct sequence with the anti-HSA VHH located at the N-terminus, the GM-CSF located at the C-terminus, and G4S as the linker.

EVQLLESGGGLVQPGGSLRLSCAASGETLDYYAIGWFRQAPGKER
EGVSCIASSGGSTNYADSVKGRFTISRDNSKNTVYLQMNSLKPED
TAVYYCAAAVLECRTWVRGYDYWGQGTQVTVSSGGGGSGGGGSGG
GGSGGGGSAPARSPSPSTQPWEHVNAIQEALRLLNLSRDTAAEMQ
ETVEVISEMFDLQEPTCLQTRLELYKQGLRGSLTKLKGPLTMMAS
HYKQHCPPTPETSCATQIITFESFKENLKDFLLVIPFDCWEPVQE.

SEQ ID NO: 8 is VHH-GMCSF construct sequence with the anti-HSA VHH located at the N-terminus, the GM-CSF located at the C-terminus, and hinge as the linker.

EVQLLESGGGLVQPGGSLRLSCAASGETLDYYAIGWFRQAPGKER
EGVSCIASSGGSTNYADSVKGRFTISRDNSKNTVYLQMNSLKPED
TAVYYCAAAVLECRTVVRGYDYWGQGTQVTVSSEPKSSDKTHTSP
PSPAPEAPARSPSPSTQPWEHVNAIQEALRLLNLSRDTAAEMQET
VEVISEMFDLQEPTCLQTRLELYKQGLRGSLTKLKGPLTMMASHY
KQHCPPTPETSCATQIITFESFKENLKDFLLVIPFDCWEPVQE.

SEQ ID NO: 9 is VHH-GMCSF construct sequence with the GM-CSF located at the N-terminus, the anti-HSA VHH located at the C-terminus, and hinge as the linker.

APARSPSPSTQPWEHVNAIQEALRLLNLSRDTAAEMQETVEVISE
MFDLQEPTCLQTRLELYKQGLRGSLTKLKGPLTMMASHYKQHCPP
TPETSCATQIITFESFKENLKDFLLVIPFDCWEPVQEEPKSSDKT
HTSPPSPAPEEVQLLESGGGLVQPGGSLRLSCAASGETLDYYAIG
WFRQAPGKEREGVSCIASSGGSTNYADSVKGRFTISRDNSKNTVY
LQMNSLKPEDTAVYYCAAAVLECRTVVRGYDYWGQGTQVTVSS.

SEQ ID NO: 10 is VHH-GMCSF construct sequence with the GM-CSF located at the N-terminus, the anti-HSA VHH located at the C-terminus, and G4S as the linker.

APARSPSPSTQPWEHVNAIQEALRLLNLSRDTAAEMQETVEVISE
MFDLQEPTCLQTRLELYKQGLRGSLTKLKGPLTMMASHYKQHCPP
TPETSCATQIITFESFKENLKDFLLVIPFDCWEPVQEGGGGSGGG
GSGGGGSGGGGSEVQLLESGGGLVQPGGSLRLSCAASGETLDYYA
IGWFRQAPGKEREGVSCIASSGGSTNYADSVKGRFTISRDNSKNT
VYLQMNSLKPEDTAVYYCAAAVLECRTVVRGYDYWGQGTQVTVSS.

SEQ ID NO: 11 is VHH-GMCSF construct sequence with the anti-HSA VHH located at the N-terminus, the GM-CSF located at the C-terminus, and G4A as the linker.

EVQLLESGGGLVQPGGSLRLSCAASGETLDYYAIGWFRQAPGKER
EGVSCIASSGGSTNYADSVKGRFTISRDNSKNTVYLQMNSLKPED
TAVYYCAAAVLECRTVVRGYDYWGQGTQVTVSSGGGGAGGGGAGG
GGAGGGGAAPARSPSPSTQPWEHVNAIQEALRLLNLSRDTAAEMQ
ETVEVISEMFDLQEPTCLQTRLELYKQGLRGSLTKLKGPLTMMAS
HYKQHCPPTPETSCATQIITFESFKENLKDFLLVIPFDCWEPVQE.

SEQ ID NOs: 12-14 are CDRs: CDR-H1: GETLDYYA (SEQ ID NO: 12), CDR-H2: IASSGGST (SEQ ID NO: 13), and CDR-H3: AAAVLECRTVVRGYDY (SEQ ID NO: 14).

SEQ ID NOs: 15-17 are CDRs:CDR-H1: ETLDYYAIG (SEQ ID NO: 15), CDR-H2: IASSGGSTNYADSVKG (SEQ ID NO: 16), CDR-H3: AVLECRTVVRGYDY(SEQ ID NO: 17).

### Definitions

The following definitions are used in connection with the invention disclosed herein. Unless defined otherwise, all technical and scientific terms used herein have the same meaning as commonly understood to one of skill in the art to which this invention belongs.

An “effective amount,” when used in connection with an agent effective for the treatment of a coronavirus infection is an amount that is effective for treating or mitigating a coronavirus infection.

As used herein, “a,” “an,” or “the” can mean one or more than one. Further, the term “about” when used in connection with a referenced numeric indication means the referenced numeric indication plus or minus up to 10% of that referenced numeric indication. For example, the language “about 50” covers the range of 45 to 55.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

This invention is further illustrated by the following non-limiting examples.

### EXAMPLES

### Example 1: Discovery and Humanization of the VHH Molecule

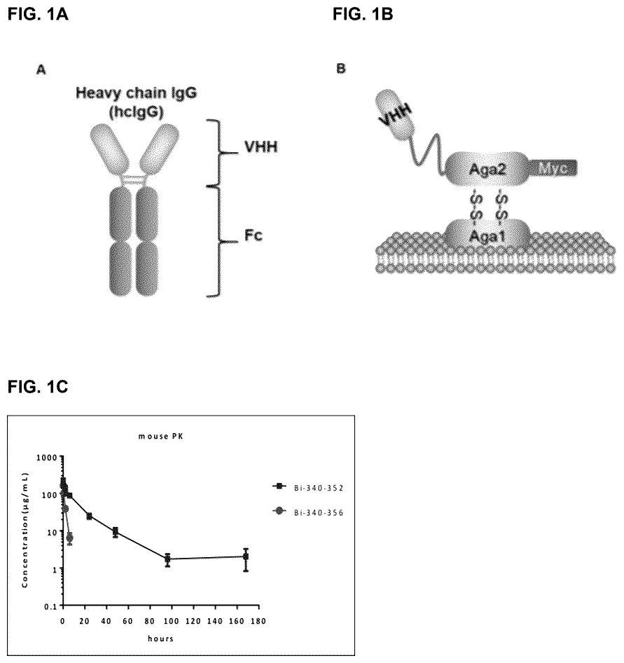

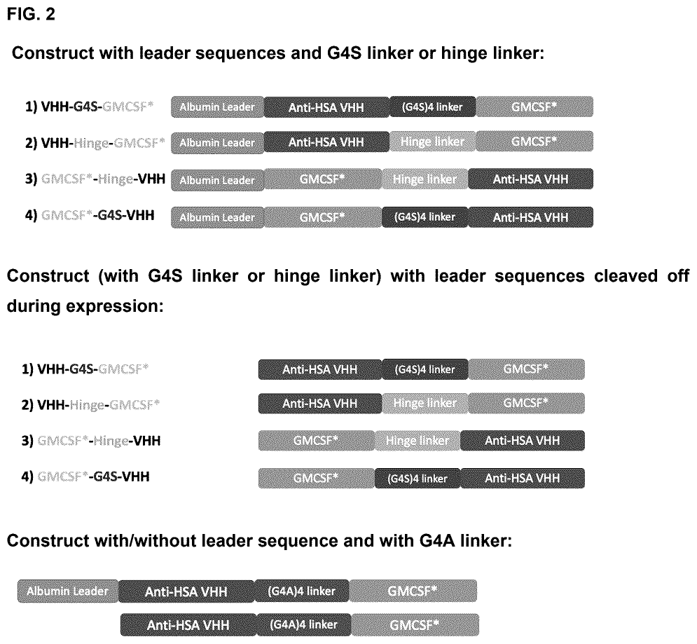

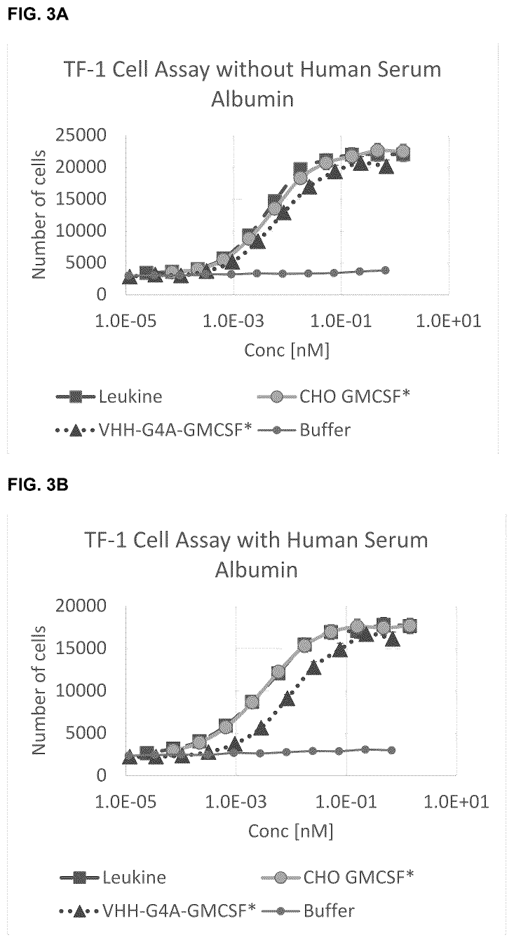

Two Llamas were immunized with human serum albumin (HSA) to generate antibodies. Total RNA was isolated from cells, followed by cDNA synthesis. Using this cDNA, the variable domain of the heavy chain for the camelid heavy-chain only antibody (VHH) encoding sequences were amplified by PCR. A structure representation of heavy chain IgG (hclgG) is shown in FIG. 1A.

Subsequently, fluorescently activated cell sorting (FACs) in combination with fluorescently labeled HSA to bind to yeast expressing a VHH that binds human serum albumin (HSA) was used to isolate individual VHH that bind HSA. The yeast surface display system for the screening of antigen-binding scaffold libraries is shown in FIG. 1B. These VHH also did not bind albumin at an epitope that would block FcRn binding which is important for the recycling and pharmacokinetic (pK) properties of albumin. The clones were also shown not to bind non-specifically to a membrane preparation. Lastly, the binding properties of the VHH clones did not change when measured between pH 5.5 and 7.4. Following identification of the HSA binding VHH clones, the VHH was humanized by CDR grafting into an appropriate human VHH with appropriate back mutations and Llama specific sequences required to maintain VHH stability.

The three anti-HSA VHH clones identified and humanized were used in an experimental fusion protein to demonstrate the VHH facilitates an increase in serum half-life. Protein fusion molecules were made to investigate the potential half-life extension capabilities of the three VHHs. A Fab molecule fusion protein was constructed with each of the three VHHs. Half-life extension of a Fab fragment facilitated by VHH was demonstrated in a murine model demonstrates 17 fold increase in half-life of the Fab. VHH59-1 also more than doubles the half-life of other anti-HSA-VHH molecules (FIG. 1C).

### Example 2: Identification and Construction of GMCSF-VHH

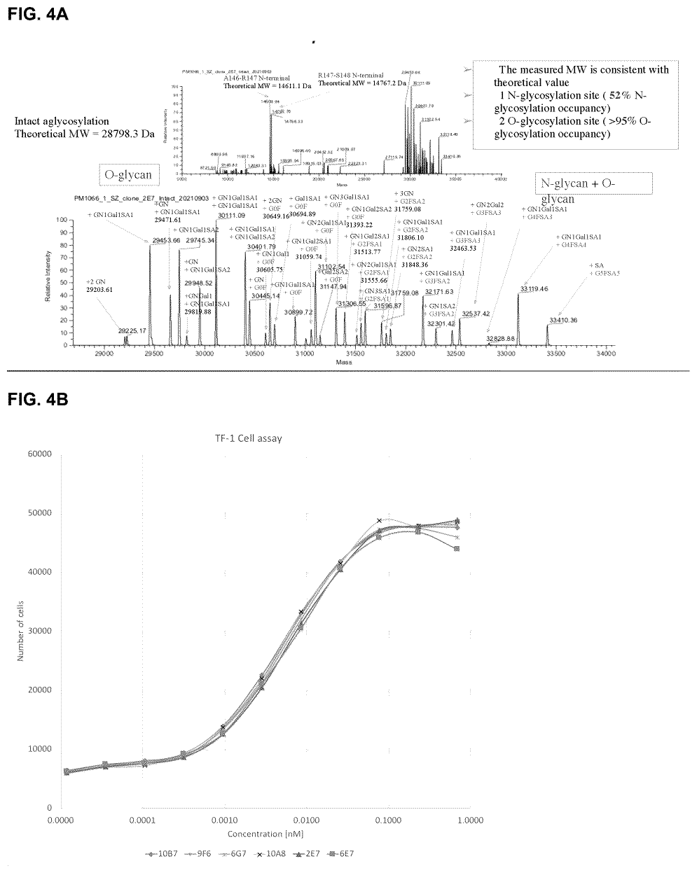

Four variants were initially made and characterized. The orientation of the VHH and GMCSF were varied around two linker sequences: VHH-linker-GMCSF or GMCSF-linker-VHH. The (GGGGS)x4 or (GGGGA)x4 linker were used. The second sequence is the modified hinge sequence of human IgG1 as the VHH is linked directly to the hinge region of an IgG in the llama, i.e. a link between the VHH and the constant region of the natural antibody in llamas. The albumin leader sequence was chosen over the native GMCSF leader as data suggests the cleavage of the leader is heterogeneous when assessed using SignalP software analysis whereas the albumin leader is homogeneously cleaved. The 4 constructs were cloned and transiently expressed in CHO cells. The secretion leader sequence was the same for all 4 constructs. The sequences are shown in FIG. 2.

### Example 3: Characterization of the VHH-GA-GMCSF* Molecule

Multiple variations of the anti-HSA-VHH59-1 were made so that we could empirically determine which variant had the best developability characteristics including expression titers, aggregation propensity as determined by size exclusion chromatography, ability of the VHH to bind HSA within the context of a fusion or chimeric protein, bio activity of the fused GMCSF and binding characteristics of the GMCSF to its cognate receptor. The *GM-CSF sequence used to make the fusion was identical to the GMCSF variant sequence previously used to express the CHO-derived GM-CSF variant.

Expression titers levels in transient CHO for the 4 constructs are shown in Table 1. Expression level for GM-CSF at N-terminus was much higher than at the C-terminus. A 30% loss in yield was seen due to the relatively weak binding between VHH and Pro A resin. However, this GMCSF-Hinge-VHH also appeared to contains more aggregates as compared with other constructs.

TABLE 1

| | | Pro A | | | | |
|---|---|---|---|---|---|---|
| | Fortebio | flowthru | Yield | Calculated yield | | |
| | Titer | Titer | percentage | based on Fortebio | Actual yield | Yield |
| Sample | (mg/L) | (mg/L) | by Pro A | titer (mg) | (mg) | percentage |
| VHH-(G4S)₄ linker-GMCSF* | 102.0 | 27 | 74% | 30.6 | 44.5 | 145% |
| VHH-Hinge linker-GMCSF+ | 65.9 | 20.4 | 69% | 19.8 | 22.8 | 115% |
| GMCSF*-Hinge linker-vHH | 209.4 | 59.8 | 71% | 62.8 | 60.0 (10% aggregates) | 96% |
| GMCSF*-(G4S)₄ linker-vHH | 182.7 | 59.2 | 68% | 54.8 | 86.2 | 157% |

The ability of the VHH portion of the fusion or chimeric protein to bind to human serum albumin (HSA) using a ForteBio based assay was assessed. Interestingly, all 4 of the VHH-GMCSF fusion or chimeric proteins were able to bind HSA only when they were immobilized on the sensor, and the albumin was in solution, but not when the albumin was mobilized on the sensor.

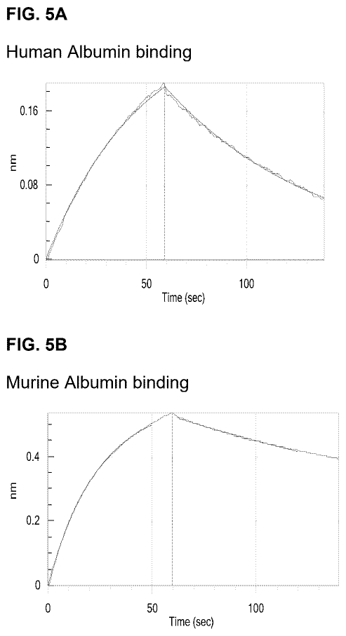

The ability of the GM-CSF within the fusion or chimeric to cognate receptor was assessed in a bioactivity assay using TF-1 cells in the presence (FIG. 3A) and absence of albumin (FIG. 3B). TF-1 cells proliferated in response to exposure of the fusion or chimeric GM-CSF. The four chimeric or fusion proteins were compared to constructs that contained CHO-derived GMCSF* variant (VHH-GMCSF) as well as LEUKINE and commercial GM-CSF. The data showed that the VHH-G4Sx4-GM-CSF and GM-CSF-G4Sx4-VHH were biologically active in the TF-1 cell based assay, i.e. the cells proliferated when the GM-CSF fusion or chimeric proteins were added to the culture media. However, the GMCSF-G4Sx4-VHH EC50 (Table 2) was noted to be slightly lower, or less active, than the other orientation. The fusion or chimeric protein that contained the CHO-derived GM-CSF* showed similar activity LEUKINE. Thus the results showed that the orientation of the GM-CSF in relation to the VHH within the fusion or chimeric protein had an effect on its biologic activity in the cell based assay in both the presence and absence of albumin, with the inhibition of GM-CSF activity being greater in the presence of albumin. The albumin when bound to the VHH, without wishing to be bound by theory, potentially caused an alteration in the ability of the GM-CSF (at the N-terminus) to interact with its receptor and thus was unable to induce the TF1 cells to proliferate as compared to GM-CSF alone, or when the GMCSF was fused to the C-terminus of the VHH-linker (FIG. 3A-B).

TABLE 2

| | EC50 |
|---|---|
| Leukine | 0.003041 |
| CHO-GMCSF* | 0.00299 |
| VHH-G4A-GMCSF* | 0.009436 |
| Buffer | 0 |

### Example 4: Mass Spectrometry Assessment of the Glycan Profile of the Fusion Proteins

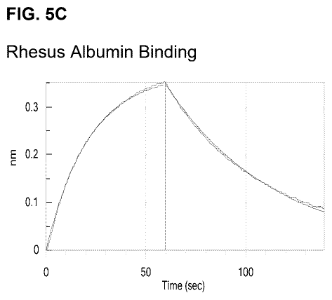

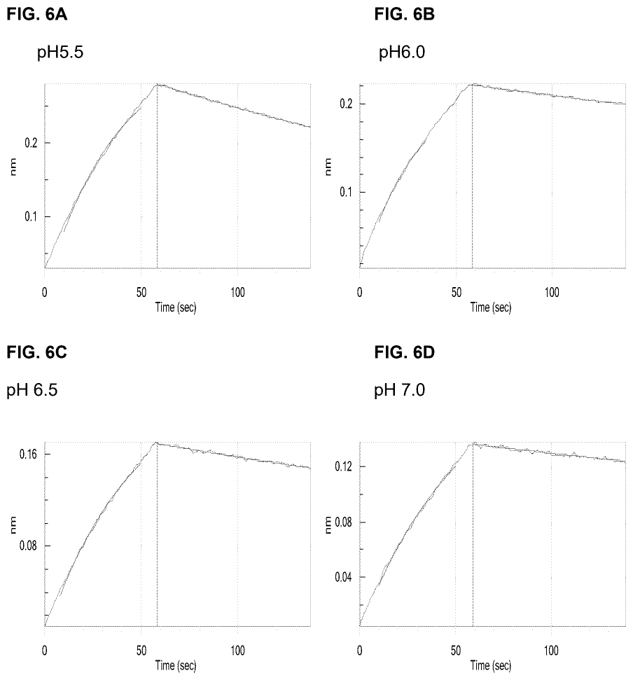

Mass spectrometry analysis was used to verify N-terminus cleavage heterogeneity as well as glycosylation of the molecule. Mass spectrometry identified the (G4Sx4) linker had the addition of xylose, which is known to be immunogenic in humans. See Arch Biochem Biophys. 426:266-278. The mass spectrometry analysis also showed that the known glycosylation sites in GM-CSF, Ser7, Ser10 and N27, were all also glycosylated in all the VHH-G4Sx4-GM-CSF variant, and were similar to the CHO-produced GMCSF* used in the fusion proteins. Six different stable lines were analyzed and results were tabulated below. High resolution liquid chromatography-mass spectrometry (LC-MS) (Vanquish UPLC Q Exactive, Thermo) was used to determine the intact molecular weight and glycan profile (FIG. 4A). The figure show glycan analysis by mass spectrometry and specifically glycan occupancy of 6 stable CHO clones making the VHH-G4A GMCSF*. 100 μg sample was diluted by deionized water to 2 mg/mL, then injected to LC-MS. Data was analyzed and deconvoluted by BioPharmaFinder software to obtain the intact protein molecular mass. The glycosylation species were identified by the precise mass shift and manual calculations. Based on the intensity of all the identified ions, the N/O glycosylation occupancy was estimated based on the assumption that their ionization efficiencies are comparable (Table 3).

TABLE 3

| Sample ID | | | |
|---|---|---|---|
| of protein | | | |
| sample | O-glycosylation | N-glycosylation | |
| analyzed | occupancy (%) | occupancy (%) | Comments |
| 2E7 | >95% | 52.2% | The measured |
| 6E7 | >90% | 46.4% | molecular weights |
| 6G7 | >90% | 27.6% | are consistent with |
| 9F6 | >80% | 14.9% | the theoretical value |
| 10A8 | >80% | 13.6% | indicating correct |
| 10B7 | >80% | 12.3% | expression. |

Constructs produced from these 6 clones were assayed in a TF-1 cell based assay. The data suggests that even though the N-linked occupancy at N27 varies between approximately 12% and approximately 53%, the activity was not altered. Thus, the activity of the molecule did not change and appeared to not be influenced by the presence or absence glycan (FIG. 4B and Table 4).

TABLE 4

| Clone | EC50 |
|---|---|
| 10B7 | 0.00479 |
| 9F6 | 0.00509 |
| 6G7 | 0.00504 |
| 10A8 | 0.00506 |
| 2E7 | 0.00612 |
| 6E7 | 0.00523 |

### Example 5: In Vitro Binding Characterization of VHH-GMCSF Constructs

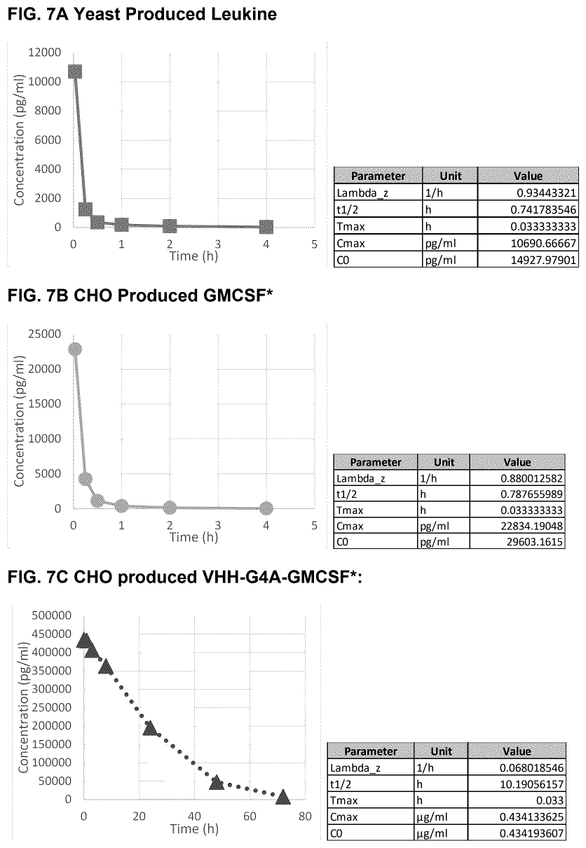

To assess binding to albumin, the VHH were cloned onto a human IgG1 Fc and hinge for ease of purification and analysis. Binding affinities of the VHH-Fc constructs were determined by BLI using an Octet 384 (Pall Life Sciences). The VH-Fc constructs were loaded onto AHC sensors prior to exposure to the monomeric targets human, mouse or rhesus albumin in solution for association and then dissociation in buffer solution (Table 5 and FIGS. 5A-C).

TABLE 5

| | Response | KD (M) | kon(1/Ms) | kdis(1/s) |
|---|---|---|---|---|
| Human Albumin | 0.1779 | 1.59E−07 | 8.22E+04 | 1.30E−02 |
| Mouse Albumin | 0.5073 | 1.04E−08 | 3.73E+05 | 3.86E−03 |
| Rhesus albumin | 0.3404 | 6.62E−08 | 2.80E+05 | 1.85E−02 |

To determine the dissociation constant, the binding reaction and dissociation were performed at the described pH. Data acquisition was performed at 30° C.

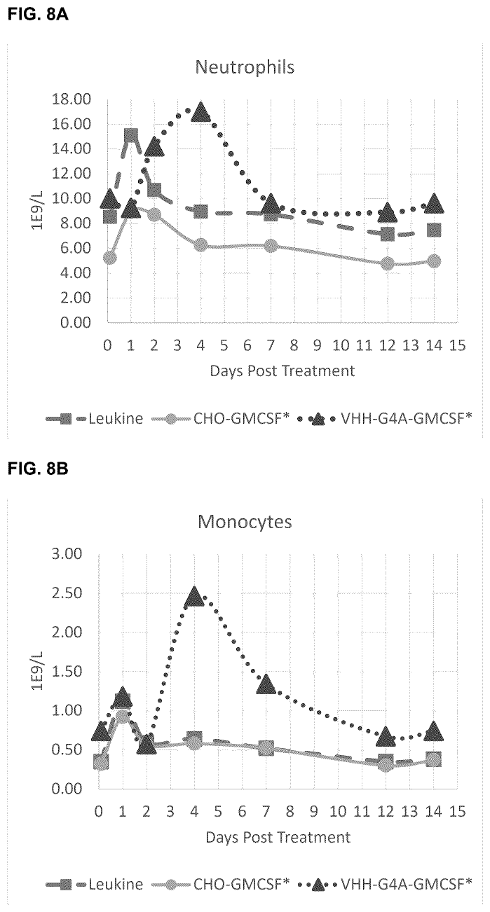

The graphs show the response units (target binding to the VHH-Fc on the chip sensor) on the Y axis and time on the X axis (FIGS. 6A-D). Data (time and response units) was analyzed using the ForteBio Data Analysis Software. The data was fit to a 1:1 binding model to calculate an association (Kon) and dissociation rate (Koff), and KD was calculated using the ratio kd/ka. Table 6 shows the measured and calculated parameters for each target and each pH.

TABLE 6

| | Response | KD (M) | kon(1/Ms) | kdis(1/s) |
|---|---|---|---|---|
| pH 5.5 | 0.2589 | 1.23E−08 | 2.41E+05 | 2.95E−03 |
| pH 6.0 | 0.2054 | 3.81E−09 | 3.42E+05 | 1.30E−03 |
| pH 6.5 | 0.1571 | 8.20E−09 | 2.07E+05 | 1.70E−03 |
| pH 7.0 | 0.1262 | 6.63E−09 | 1.78E+05 | 1.18E−03 |

### Example 6: In Vivo Characterization of VHH-GA-GMCSF*

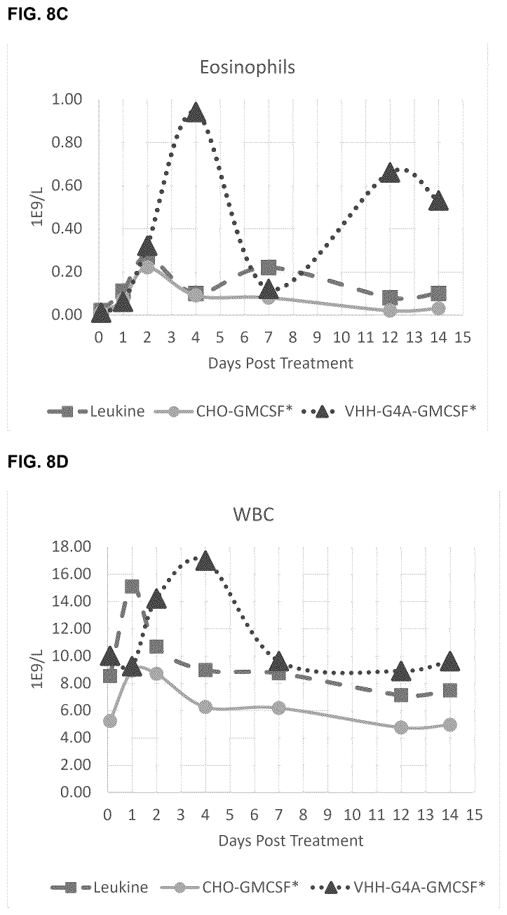

The VHH-GA-GMCSF* and two control molecules, CHO-produced GMCSF* and Leukine, were experimentally tested in a monkey study (one monkey per molecule being tested). The pK as well as the biologic effect on circulating hematopoietic cells were measured for each of the three molecules over a time course after IV injection of 20 ug for VHH-GA-GM-CSF* or 10 ug for Leukine or CHO-produced GM-CSF*. The VHH-GA-GM-CSF* was dosed at a higher concentration due to it being approximately twice the molecule weight of GM-CSF* and therefore by doubling the amount, equal molar amounts of the molecules were administered. Blood draws were taken at 12 min, 30 min, 1, 2, 4, 8, 24, 48 and 72 hours post injection. The data (FIGS. 7A-C) showed that the VHH in the VHH-GA-GMCSF* construct facilitated a significant increase in pK from 0.79 hours to >10 hours. Leukine and CHO-produced GMCSF* showed similar half-life (t_{1/2}) indicating CHO-production of GMCSF was not responsible for the half life extension. A single Rhesus monkey was intravenously injected with 10 ug/Kg of the listed form of GMCSF. Blood draws were taken at 12 min, 30 min, 1, 2, 4, 8, 24, 48 and 72 hours post injection. The blood was analyzed for cell content and the concentration of the construct in the blood was determined by a construct specific proprietary ELISA. VHH-GA-GMCSF* and shows a t_{1/2} approximately 17 fold greater than Leukine or the CHO produced form of GMCSF used in the VHH-GA-GMCSF construct.

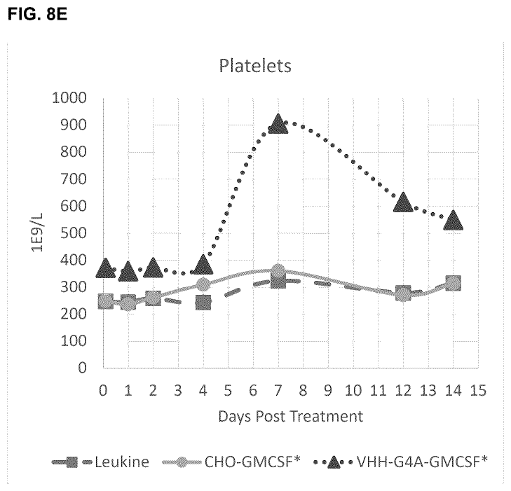

A complete hematopoietic assessment of the cell count and cell types following blood draws from the injected monkeys was performed using FACS analysis with cell specific markers (FIG. 8A-E). The numbers of neutrophils, monocytes, eosinophils, total WBC and platelets were determined (FIG. 8A-E). Both the CHO-produced GM-CSF* and LEUKINE showed a similar sharp increase in cell counts for neutrophils, monocytes and WBCs 24 hours post injection, returning to baseline by day 4. However, the neutrophil, monocyte and WBC cell counts for fusion or chimeric VHH-GA-GMCSF* peaked at day 2 and return to baseline between day 8-12. The longer duration of neutrophils and monocytes and WBCs following injection with the fusion or chimeric protein suggested the enhanced pk shown in FIGS. 7A-C resulted in a biologic readout of increased blood cells in circulation for a longer duration. Similar delayed kinetics for an increase in eosinophils were observed following injection with the fusion or chimeric protein. There was also an enhanced number of neutrophils, monocytes, eosinophils and WBCs following injection the chimeric or fusion protein. Surprisingly, the data showed a significant increase in the number of platelets following injection with the fusion or chimeric VHH-GA-GMCSF* as compared to both the CHO-produced GM-CSF* and LEUKINE.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

As used herein, all headings are simply for organization and are not intended to limit the disclosure in any manner. The content of any individual section may be equally applicable to all sections.

What is claimed is:

1. A composition comprising a fusion or chimeric protein comprising a recombinant human granulocyte macrophage-colony stimulating factor (GM-CSF) protein, a linker and a humanized single domain antibody.

2. The composition of claim 1, wherein the recombinant human GM-CSF protein comprises an amino acid sequence having at least about 97% identity with SEQ ID NO: 1 or SEQ ID NO: 2.

3. The composition of claim 2, wherein the recombinant human GM-CSF protein comprises an amino acid sequence having at least about 97% identity with SEQ ID NO: 1 or SEQ ID NO: 2 and a substitution or deletion at position N37 or a position corresponding thereto.

4. The composition of claim 3, wherein the amino acid at position N37 or a position corresponding thereto is a polar and neutral of charge hydrophilic amino acid.

5. The composition of claim 4, wherein the polar and neutral of charge hydrophilic amino acid is selected from glutamine (Q), serine (S), and threonine (T).

6. The composition of claim 5, wherein the polar and neutral of charge hydrophilic amino acid is selected from glutamine (Q), serine (S), threonine (T), proline (P), and cysteine (C).

7. The composition of claim 6, wherein the polar and neutral of charge hydrophilic amino acid is glutamine (Q).

8. The composition of claim 1, wherein the humanized single domain antibody is a variable fragment of heavy chain antibody (VHH).

9. The composition of claim 8, wherein the VHH is specific for human serum albumin.

10. The composition of claim 8 or 9, wherein the VHH comprises an amino acid sequence having at least about 80%, or at least about 85%, or at least about 90%, or at least about 95% identity with SEQ ID NO: 3 and/or comprises three CDRs selected from (i) SEQ ID NOs: 12-14, or variants thereof or (ii) SEQ ID NOs: 15-17, or variants thereof.

11. The composition of any one of claims 8-10, wherein the VHH further comprises substitutions or deletions within the framework region relative to a non-humanized VHH.

12. The composition of any one of claims 1-11, wherein the recombinant human GM-CSF is connected to the single domain antibody via a linker.

13. The composition of claim 12, wherein the linker is a flexible linker.

14. The composition of claim 13, wherein the flexible linker is substantially comprised of glycine and serine residues.

15. The composition of claim 13 or 14, wherein the flexible linker comprises at least about 20, or at least about 30, or at least about 40, or at least about 50, or at least about 60 amino acid residues.

16. The composition of any one of claims 13-15, wherein the flexible linker comprises (Gly₄Ser)ₙ, where n is from about 1 to about 12.

17. The composition of claim 12, wherein the linker is a hinge-CH2-CH3 Fc domain.

18. The composition of claim 17, wherein the hinge-CH2-CH3 Fc domain is derived from a human IgG.

19. The composition of claim 18, wherein the hinge-CH2-CH3 Fc domain is derived from human IgG1, IgG2 or IgG4.

20. The composition of claim 12, wherein the linker comprises an amino acid sequence selected from SEQ ID NO: 4, SEQ ID NO: 5, and SEQ ID NO: 6, or an amino acid sequence having at least about 80%, or at least about 85%, or at least about 90%, or at least about 95% identity with SEQ ID NO: 6 or an amino acid sequence of SEQ ID NO: 4 or SEQ ID NO: 5 having about 5, or about 4, or about 3, or about 2, or about 1 substitutions or deletions.

21. The composition of claim 20, wherein the linker comprises an amino acid sequence having at least about 80%, or at least about 85%, or at least about 90%, or at least about 95% identity with SEQ ID NO: 6.

22. The composition of any one of claims 1-21, wherein the VHH domain is positioned at the N-terminus of the fusion or chimeric protein.

23. The composition of any one of claims 1-21, wherein the GM-CSF is positioned at the N-terminus of the fusion or chimeric protein.

24. The composition of any of claims 1-23, wherein the fusion or chimeric protein comprises an amino acid sequence selected from SEQ ID NO: 7, SEQ ID NO: 8, SEQ ID NO: 9, SEQ ID NO: 10, and SEQ ID. NO: 11, or an amino acid sequence having at least about 80%, or at least about 85%, or at least about 90%, or at least about 95% identity with SEQ ID NO: 7, SEQ ID NO: 8, SEQ ID NO: 9, SEQ ID NO: 10, or SEQ ID NO: 11.

25. The composition of any of claims 1-24, wherein the composition binds and/or activates the granulocyte-macrophage colony stimulating factor receptor (GM-CSF-R-alpha or CSF2R).

26. The composition of claim 25, wherein the GM-CSF-R-alpha is expressed on the surface of a cell.

27. The composition of claim 26, wherein the cell is a hematopoietic progenitor cell.

28. The composition of claim 27, wherein the hematopoietic progenitor cell is an immune cell.

29. The composition of claim 27, wherein the hematopoietic progenitor cell is irradiated.

30. The composition of any of the claims 1-29, wherein the composition binds human, mouse and rhesus serum albumin.

31. The composition of claim 30, wherein the composition binds human serum albumin (HSA).

32. The composition of claim 31, wherein the HSA is present in human blood plasma.

33. The composition of claim 32, wherein the composition does not substantially compete with HSA binding to neonatal Fc receptor (FcRn).

34. The composition of any of the above claims, wherein the fusion or chimeric protein is soluble.

35. The composition of any of the above claims, wherein fusion or chimeric protein demonstrates substantially similar functionality as sargramostim.

36. The composition of any of the above claims, wherein the recombinant human GM-CSF comprises a plurality of molecular forms.

37. The composition of claim 36, wherein the molecular forms are selected from non-glycosylated, O-glycosylated, N-glycosylated and N+O glycosylated forms.

38. The composition of any of the above claims, wherein the recombinant human GM-CSF is substantially free of hypermannosylated forms.

39. A nucleic acid molecule encoding the fusion or chimeric protein of any of the above claims, or a fragment thereof.

40. The nucleic acid of claim 39, wherein the nucleic acid molecule has a codon-optimized sequence, or a component thereof.

41. A human host cell expressing the nucleic acid molecule of claim 39 or 40.

42. The host cell of claim 41, wherein the host cell is a Chinese Hamster Ovary (CHO) cell.

43. A non-human host cell expressing the nucleic acid molecule of claim 39 or 40.

44. The non-human host cell of claim 43, wherein the host cell is a yeast cell.

45. The non-human host cell of claim 44, wherein the yeast cell is a non-methylotrophic yeast cell.

46. The non-human host cell of claim 45, wherein the host cell is a Saccharomyces cerevisiae cell.

47. A pharmaceutical composition comprising a composition of any one of claims 1-38 and a pharmaceutically acceptable excipient or carrier.

48. A method of treating a patient or subject who is undertaking or has undertaken a cancer therapy, or who is undertaking or has undertaken a bone marrow transplant, or who had been acutely exposed to myelosuppressive doses of radiation (e.g. H-ARS), and/or who has a Radiation Combined Injury (RCI); the method comprising administering to the patient a therapeutically effective amount of the pharmaceutical composition of claim 47.

49. The method of claim 48, wherein the patient is treated by modulating clonal expansion, survival, differentiation and activation state of hematopoietic progenitor cells.

50. The method of claim 48, wherein the patient is treated by modulating a myelomonocytic cell lineage, by promoting the proliferation of megakaryocytic and erythroid progenitors.

51. The method of claim 48, wherein the patient is treated by modulating hematopoietic progenitor cells, by stimulating the survival, proliferation and activation of neutrophils, macrophages and/or dendritic cells.

52. The method of claim 48, wherein the patient is treated following bone marrow transplant by modulating hematopoietic progenitor cells, by stimulating the survival, proliferation and activation of neutrophils, macrophages and/or dendritic cells.

53. A method of therapy, comprising
administering to a patient a therapeutically effective amount of the pharmaceutical composition of claim 47 or
contacting cells with an effective amount of the pharmaceutical composition of claim 47 and administering therapeutically effective amount of the cells, wherein the therapy:
accelerates neutrophil recovery and/or reduces the incidence of infections following induction chemotherapy;
mobilizes hematopoietic progenitor cells into peripheral blood for collection by leukapheresis and transplantation;
accelerates of myeloid reconstitution following autologous or allogeneic bone marrow or peripheral blood progenitor cell transplantation;
treats delayed neutrophil recovery or graft failure after autologous or allogeneic bone marrow transplantation; and/or
treats hematopoietic syndrome of Hematopoietic Acute Radiation Syndrome (H-ARS);
treats Radiation Combined Injury (RCI);
treats a neurodegenerative disease;
treats nerve transection;
treats traumatic brain injury;
treats a pulmonary infection;
treats an autoimmune disease, optionally Crohn's or IBD;
treats sepsis/septicemia;
treats an invasive fungal infection;
treats multiple organ dysfunction syndrome (MODS);
treats peripheral artery disease/peripheral arterial disease/cardiovascular disease;
treats an oncology indication
facilitates wound healing and/or treats a dermatological condition.

54. A method for treating an infection with a coronavirus, comprising: administering an effective amount of the pharmaceutical composition of claim 47 to a patient in need thereof.

55. The method of claim 54, wherein the coronavirus is a betacoronavirus, optionally selected from severe acute respiratory syndrome coronavirus 2 (SARS-CoV-2), SARS-CoV, Middle East respiratory syndrome-corona virus (MERS-CoV), HCoV-HKU1, and HCoV-OC43.

56. The method of claim 55, wherein the coronavirus is an alphacoronavirus, optionally selected from HCoV-NL63 and HCoV-229E.

57. The method of claim 56, wherein the coronavirus is SARS-CoV-2.

58. The method of claim 56, wherein the patient is afflicted with COVID-19.

59. A method for treating an infection with influenza virus, optionally selected from Type A, Type B, Type C, and Type D, comprising: administering an effective amount of the pharmaceutical composition of claim 47 to a patient in need thereof.

60. A method of treating Viral Hemorrhagic Fevers, caused by a group of viruses optionally selected from Arenaviruses, Flaviviruses, Filoviruses, Hantaviruses, Nairoviruses, Phenuviruses, comprising: administering an effective amount of the pharmaceutical composition of claim 47 to a patient in need thereof.

61. A method of treating a lung infection, where the lung infection is Pneumonic Plague caused by an infection with Yersinia pestis, comprising: administering an effective amount of the pharmaceutical composition of claim 47 to a patient in need thereof.

62. The method of any one of claims 54-61, wherein the patient is afflicted with one or more of fever, cough, shortness of breath, diarrhea, upper respiratory symptoms, lower respiratory symptoms, pneumonia, and acute respiratory syndrome.

63. The method of any one of claims 54-62, wherein the patient is hypoxic.

64. The method of any one of claims 54-63, wherein the patient is afflicted with respiratory distress.

65. The method of any one of claims 54-64, wherein the method prevents or mitigates development of acute respiratory distress syndrome (ARDS) in the patient.

66. The method of any one of claims 54-65, wherein the method improves oxygenation in the patient.

67. The method of any one of claims 54-66, wherein the method prevents or mitigates a transition from respiratory distress to cytokine imbalance in the patient.

68. The method of any one of claims 54-67, wherein the method reverses or prevents a cytokine storm.

69. The method of claim 68, wherein the method reverses or prevents a cytokine storm in the lungs or systemically.

70. The method of claim 68 or 69, wherein the cytokine storm is selected from one or more of systemic inflammatory response syndrome, cytokine release syndrome, macrophage activation syndrome, and hemophagocytic lymphohistiocytosis.

71. The method of claim 70, wherein the method reverses or prevents excessive production of one or more inflammatory cytokines.

72. The method of claim 71, wherein the inflammatory cytokine is one or more of IL-6, IL-1, IL-1 receptor antagonist (IL-1ra), IL-2ra, IL-10, IL-18, TNFα, interferon-γ, CXCL10, and CCL7.

73. The method of any one of claims 54-72, wherein the method causes a decrease in viral or bacterial load in the patient relative to before treatment.

74. A method of treating and/or ameliorating symptoms caused by exposure to chemical warfare agents, comprising: administering an effective amount of the pharmaceutical composition of claim 47 to a patient in need thereof.

75. A method of claim 74, wherein the chemical warfare agents is Sulphur Mustard gas (SM/HD) and/or chlorine gas.

76. The method of claim 74 or 75, where the symptoms of exposure in the patient is characterized by myelosuppression.

77. The method of any one of claims 74-76, wherein the composition causes an amelioration of chemical exposure symptoms, including myelosuppression.

78. A method of treating and/or ameliorating symptoms caused by a neurodegenerative disease, comprising: administering an effective amount of the pharmaceutical composition of claim 47 to a patient in need thereof.

79. The method of claim 78, wherein the neurogenerative disease is selected from Alzheimer's disease, Parkinson's disease, progressive supranuclear palsy (PSP), multiple system atrophy (MSA), Lewy body dementia, Parkinson's disease dementia epilepsy, stroke, Huntington's Chorea, cerebral hypoxia, multiple sclerosis, amyotrophic lateral sclerosis (ALS), and peripheral neuropathy.

80. The method of claim 78 or 79, wherein the neurodegenerative disease is characterized by oxidative stress, loss of neurite integrity, apoptosis, neuronal loss or/and inflammation response.

81. The method of any one of claims 78-80, wherein the neurodegenerative disease is associated with cognitive impairment.

82. The method of any one of claims 78-81, wherein the composition elicits a disease-modifying response.

83. The method of any one of claims 78-82, wherein the composition elicits temporarily or permanently slows down cognitive decline.

84. The method of any one of claims 78-83, wherein the composition causes an amelioration of neurodegenerative disease symptoms.

85. The method of any one of claims 78-84, wherein the composition slows the onset and/or development of the neurodegenerative disease.

86. The method of treating an autoimmune disease, comprising: administering an effective amount of the pharmaceutical composition of claim 47 to a patient in need thereof, wherein the autoimmune disease is optionally selected from Crohn's disease and/or ulcerative colitis (UC) and/or irritable bowel disease (IBD).

87. The method of claim 86, wherein the composition restores the balance of effector and regulatory immune function.

88. A method of treating and/or ameliorating symptoms caused by a caspase recruitment domain family member 9 (CARD9) deficiency, comprising: administering an effective amount of the pharmaceutical composition of claim 47 to a patient in need thereof.

89. The method of claim 88, wherein the method treats an invasive fungal infection.

90. The method of claim 89, wherein the invasive fungal infection is selected from candidiasis, mucormycosis, invasive aspergillosis, blastomycosis, candidiasis, coccidioidomycosis, basidiobolus, conidiobolus, cryptococcal meningitis, histoplasmosis.

91. The method of claim 90, wherein the candidiasis is esophageal candidiasis, oropharyngeal candidiasis, invasive candidiasis and/or candidemia.

92. The method of claim 90, wherein the mucormycosis is cutaneous mucormycosis.

93. The method of any one of claims 88-92, wherein the composition is used as an adjunctive therapy to anti-fungal therapies.

94. The method of any one of claims 88-93, wherein the composition aids in the decrease and/or reduction of the fungal load in various tissues.

95. A method of treating and/or facilitating wound healing and/or a dermatological condition, comprising: administering an effective amount of the pharmaceutical composition of claim 47 to a patient in need thereof.

96. The method of claim 95, wherein the chronic wound is optionally selected from lepra, leg ulcers, and/or indolent wounds of various other causes.

97. The method of claim 96, wherein the method promotes tissues remodeling and regeneration.

98. A method of treating cancer, comprising: administering an effective amount of the pharmaceutical composition of claim 47 to a patient in need thereof.

99. The method of claim 98, wherein the composition causes an immune modulation or activation the immune response against tumor cells.

100. The method of claim 98 or 99, wherein the composition enhances or restores the activity or activation of one or more cells selected from the group consisting of: T cells, T helper cells, cytotoxic T cells, dendritic cells, NK cells, macrophages, anti-tumor macrophages and B cells.

101. The method of claims 98-100 where the composition treats, ameliorates, and/or prevents cancer growth, survival, metastasis, epithelial-mesenchymal transition, immunologic escape or recurrence.

102. The method of treating sepsis or septicemia, comprising administering an effective amount of the pharmaceutical composition of claim 47 to a patient in need thereof.

103. The method of claims 98-101, wherein the composition balances and/or regulates inflammation and acute phase response, and to reduce morbidity and mortality rates.

104. The method of treating immune thrombocytopenia (ITP) or low platelet count comprising administering an effective amount of the pharmaceutical composition of claim 47 to a patient in need thereof.

105. The method of claim 103, wherein the composition boosts or enhances platelet cell count and/or reverses thrombocytopenia.

106. The composition of any one of claims 1-38, wherein the composition promotes the delivery of recombinant GM-CSF through the endothelial capillaries (blood brain barrier, BBB) for central nervous system (CNS) therapeutics.

107. The composition of any one of claims 1-38, wherein the fusion or chimeric protein is produced by transfection of a mammalian cell optionally a CHO cell, wherein the fusion or chimeric protein shows increased half-life as compared to sargramostim.

108. The composition of any one of claims 1-38, wherein the fusion or chimeric protein demonstrates enhanced pharmacokinetics as compared to sargramostim.

109. The composition of any one of claims 1-38, wherein the fusion or chimeric protein demonstrates substantially similar functionality as sargramostim.

110. The composition of any of claims 1-38, wherein the fusion or chimeric protein significantly increases the level, cell count, and/or amount of neutrophils, monocytes, eosinophils, total WBCs and platelets.

111. The composition of any of claims 1-38, wherein the fusion or chimeric protein significantly increases the level, cell count, and/or amount of platelets.

112. The composition of any one of claims 1-38, wherein the fusion or chimeric protein is produced by transfection of a mammalian cell optionally a CHO cell, wherein the chimeric protein shows improved pharmacodynamics as compared to sargramostim.

113. The composition of any one of claims 1-38, wherein the fusion or chimeric protein is produced by transfection of a mammalian cell optionally a CHO cell, wherein the fusion or chimeric protein shows similar function and improved pharmacokinetics and pharmacodynamics as compared to sargramostim.
